(19) United States
(12) Patent Application Publication
(10) Pub. No.: US 2023/0033651 A1
(43) Pub. Date: Feb. 2, 2023

(54) MICROFLUIDIC SYSTEMS AND METHODS FOR LOW-SHEAR ISOLATION OF RARE CELLS FROM LARGE SAMPLE VOLUMES

(71) Applicant: The General Hospital Corporation, Boston, MA (US)

(72) Inventors: Mehmet Toner, Charlestown, MA (US); Avanish Mishra, Dorchester, MA (US); Jon F. Edd, Wakefield, MA (US)

(21) Appl. No.: 17/790,535

(22) PCT Filed: Jan. 8, 2021
(86) PCT No.: PCT/US2021/012758

§ 371 Date: Jul. 1, 2022

Related U.S. Application Data
(60) Provisional application No. 62/958,514, filed on Jan. 8, 2020.

(51) Int. Cl. B01L 3/00; G01N 1/40

(52) U.S. Cl. CPC B01L 3/502761; G01N 1/40; B01L 2200/0652; B01L 2300/0864; B01L 2400/086; B01L 2300/0816; B01L 2400/043

(57) ABSTRACT

Systems, methods, and techniques are disclosed herein for isolating rare cells and clusters of cells, such as CTCs, from large volumes of sample fluids, such as whole blood, diluted blood, e g, minimally diluted blood, and other samples such as leukapheresis and aphaeresis samples. In some implementations, a microfluidic device includes a particle enrichment module and a particle separation module for iterative multistage sorting. Each module can have an array of islands in a microfluidic channel having a sample inlet at a first end of the first microfluidic channel. The array of islands is arranged in one or more rows that extend along a longitudinal direction in the microfluidic channel. Each island in a row is spaced apart from an adjacent island in the row to form a siphoning channel. The array of islands is configured and arranged to shift portions of fluid through the siphoning channel between adjacent islands.

### CROSS-REFERENCE TO RELATED APPLICATIONS

This application claims the benefit of the U.S. Provisional Patent Application No. 62/958,514 filed Jan. 8, 2020, which is incorporated herein by reference in its entirety.

This invention was made with government support under P41EB002503 and U01EB012493 awarded by the National Institute of Biomedical Imaging and Bioengineering and U01CA214297, 2R01CA129933, and R01CA226871 awarded by the National Cancer Institute, and 132030-RSG-18-108-01-TBG awarded by the American Cancer Society. The government has certain rights in the invention.

### TECHNICAL FIELD

This disclosure relates to the isolation of rare cells and/or rare cell clusters, for example, circulating tumor cells (CTCs) or circulating tumor cell clusters (CTCCs).

### BACKGROUND

CTCs seed metastasis by traveling through the peripheral blood of cancer patients, making them key actors in cancer progression and an important liquid biopsy target for personalized oncology. Circulating tumor cell clusters (CTCCs) have been inferred as fifty-fold more metastatic than CTCs, and they have distinct epigenetic markers. Because CTCCs enter the bloodstream with their nearest neighbors from the tumor, they are hypothesized to be easier to culture for drug testing. However, they can be weakly bound to each other and are even rarer than single CTCs. Isolating CTCCs from large blood volumes can unlock a unique reservoir of biomarkers linked to a most metastatic population of tumor cells, and it can also enable frequent, minimally-invasive sampling of the CTCC genome, transcriptome, and proteome, and in vitro drug testing on patient-specific cell lines.

However, CTCC isolation is a major technical challenge. Even from metastatic patients, there may be only 1-2 clusters in a tube of blood (7.5 mL nominal volume). Thus, it is necessary to sift through multiple tubes of blood to achieve a reliable CTCC-based assay. Assuming one analyzes four tubes of blood, there would be about 150 billion erythrocytes (RBCs), 9 billion platelets, and 180 million leukocytes (WBCs), which would need to be removed to a high degree without losing any CTCCs.

### SUMMARY

The present disclosure features systems and methods for isolating CTCs and CTCCs from large volumes (e.g., 100-200 mL) of sample fluids, such as whole blood, diluted blood, e.g., minimally diluted blood, and other samples such as leukapheresis and aphaeresis samples. The systems include microfluidic features that remove CTCs and CTCCs with a high degree of accuracy while minimizing loss during the isolation process. For example, separation can be rapid to avoid drift or degradation of CTCC-derived molecular signatures, yet gentle to avoid breaking clusters and uncoupling their linked bioinformatic signatures.

The systems disclosed herein include a size-based sorting microfluidic device and an immunomagnetic sorting microfluidic device that utilize different techniques to isolate rare cells from whole blood. The systems can be configured to recover rare cells from samples with high throughout and purity.

The systems described herein feature various advantages and benefits over other techniques sometimes used for isolation of CTCs and CTCCs. For example, the size-based cell sorting microfluidic device can include non-equilibrium inertial separation array (NISA) channels to assist with separation of CTCCs from whole blood. NISA modules can be placed serially to enable an interactive multistage sorting technique that improves product purity about ten-fold by removing most of the blood upstream and increases blood throughput. The NISA array design can incorporate siphoning channels that mitigate roller vortices within sample fluids at higher Reynolds numbers, which otherwise act to mix small cells/particles, leading to increased carryover into the product lane or lanes of the systems. As described herein, a siphoning channel can be appropriately sized using, for example, a specified length-to-width ratio of the siphoning channels. The dimensions of the siphoning channels allows rapid, gentle, size-sorting of CTCs and CTCCs from blood. For example, the width of separation channels is between 50 μm and 200 μm (e.g., 100 μm width), and the height can be between 100 μm and 400 μm (e.g., 150, 151, 152, 153, 154, 155, 156, 157, 158, or 159 μm height). Additionally, array parameters, such as island length (e.g., 200-800 μm, such as 450 μm), flow shift percentage (e.g., between 2-10%, such as between 3-4%) can also be specified so that size cutoff rises to between the largest leukocytes and smallest CTCCs (or large single CTCs) to approximately 15 μm. In this manner, wall shear stress falls by a factor of seven to less than 15 Pa in six-fold larger cross-section channels to enable larger CTCCs to pass.

The systems and methods disclosed herein can also improve cancer diagnostics and enable personal oncology. For example, the ability to recover more CTCs from large volume of apheresis samples can allow a higher success rate at ex-vivo culture of the CTCs, which can then be used for drug testing and can provide personalized guidance to patients. Additionally, the ability to handle large volume samples can enhance sensitivity of the existing cancer diagnostic assays based on CTCs and will enable early stage cancer diagnostics.

The immunomagnetic sorting microfluidic devices disclosed herein can be used as standalone positive- or negative-selection magnetic sorters for a diverse array of cell types, and can provide high purity and high yield isolation of the desired cells.

In one general aspect, the disclosure provides microfluidic devices that can be used for size-based sell sorting of rare cells as described herein. The microfluidic devices include a first particle enrichment module that includes a first microfluidic channel having a sample inlet at a first end of the first microfluidic channel. The first microfluidic channel is configured to shift particles above a specific size to a first product outlet at a second end of the first microfluidic channel.

The microfluidic devices also include a particle separation module that includes an array of islands in a second microfluidic channel, a buffer inlet in fluid communication with a first end of the second microfluidic channel, and a product inlet in fluid communication with the first product outlet and the first end of the second microfluidic channel. The array of islands is arranged in one or more rows that extend along a longitudinal direction in a corresponding microfluidic channel. Each island in a row is spaced apart from an adjacent island in the row to form a siphoning channel. The array of islands is configured and arranged to shift portions of fluid through the siphoning channel between adjacent islands within a row to a waste outlet, and to shift particles above a specific size into a buffer flowing in the second microfluidic channel and to a second product outlet. Additionally, the first particle enrichment module and the particle separation module are serially arranged such that a sample fluid having particles above the specific size flows from the first particle enrichment module to the particle separation module.

One or more implementations of these devices and systems include the following optional features. In some implementations, the microfluidic device includes a second particle enrichment module. The second particle enrichment module includes a set of microfluidic channels that are each configured to shift particles above the specific size to first product outlets at respective second ends of the set of microfluidic channels.

In some implementations, the first particle enrichment module includes a first array of islands in the first microfluidic channel. The first array of islands is arranged in one or more rows that extend along a longitudinal direction in the first microfluidic channel. Each island in a row is spaced apart from an adjacent island in the row to form a siphoning channel. The first array of islands is configured and arranged to shift portions of fluid through the siphoning channel between adjacent islands within a row to a waste outlet at a second end of the first microfluidic channel, and to shift particles above a specific size to a first product outlet at the second end of the first microfluidic channel. In such implementations, the second particle enrichment module includes multiple arrays of islands in a corresponding microfluidic channel each having a sample inlet at a first end of the corresponding microfluidic channel. Each array included in the multiple arrays of islands is arranged in one or more rows that extend along a longitudinal direction in a corresponding microfluidic channel. Each island in a row is spaced apart from an adjacent island in the row to form a siphoning channel. Each array included in the multiple arrays of islands is configured and arranged to shift portions of fluid through the siphoning channel between adjacent islands within a row to a waste outlet at a second end of the corresponding microfluidic channel, and to shift particles above a specific size to first product outlets at the second end of the corresponding microfluidic channel. The first particle enrichment module and the second particle enrichment module are serially arranged such that a sample fluid flows from the second particle enrichment module to the first particle enrichment module.

In some implementations, the multiple arrays of islands include two, three, four, five or more arrays of islands. In some implementations, the four arrays of islands are each arranged in parallel such that a different portion of the sample fluid introduced into the microfluidic device flows through each of the four arrays of islands.

In some implementations, each island included in the array of islands has a width between 150 and 250 μm, a length between 200 and 800 μm, and a height between 100 and 200 μm.

In some implementations, each island included in the array of islands has a length-to-width ratio greater than 1.25.

In some implementations, the siphoning channels formed in the array of islands have a respective channel width of 50 μm.

In another general aspect, the disclosure provides microfluidic devices that can be used for immunomagnetic sorting of rare cells as described herein. The microfluidic devices include a sorting channel arranged in a substrate and configured to flow a fluid sample comprising magnetized target entities. The microfluidic devices also include a magnet placed underneath the substrate. Additionally, the microfluidic devices include a permeability channel adjacent to a first side of the sorting channel. The magnet and the set of magnetic permeability particles are configured to generate a deflecting magnetic field that causes a subset of magnetized target entities in the sorting channel to be deflected away from the first side of the sorting channel.

One or more implementations of these devices and systems include the following optional features. In some implementations, the microfluidic devices include a second particle enrichment module. For example, in some implementations, the set of magnetic permeability particles are configured to increase a gradient of the deflecting magnetic field.

In some implementations, the set of magnetic permeability particles are configured to change a direction of force exerted by the deflecting magnetic field on the magnetized target entities.

In some implementations, the deflecting magnetic field causes a second subset of magnetized target entities in the sorting channel to be deflected towards a second side of the sorting channel. In such implementations, the microfluidic device further includes a second permeability channel adjacent to the second side of the sorting channel opposite to the first side of the sorting channel. The second permeability channel includes a second subset of magnetic permeability particles.

In some implementations, the microfluidic devices further include a first collection channel extending from the first side of the sorting channel such that the subset of magnetized target entities flows from the sorting channel to the first collection channel. The microfluidic devices also include a second collection channel extending from the second side of the sorting channel such that the second subset of magnetized target entities flow from the sorting channel to the second collection channel.

In some implementations, the microfluidic devices further include a magnetic permeability strip placed underneath the sorting channel in the substrate adjacent to the magnet. The magnetic permeability strip extends longitudinally along a direction of fluid flow in the sorting channel. The magnetic permeability strip is configured to intensify the gradient of the deflecting magnetic field.

In some implementations, the microfluidic devices further include a second sorting channel in the substrate. The second sorting channel is configured to receive a portion of the sample fluid flowing from the sorting channel.

In some implementations, the microfluidic devices further include an inertial focusing channel in the substrate and comprising a set of asymmetric serpentine segments. The inertial focusing channel is connected to the sorting channel such that a portion of the sample fluid flows from the inertial focusing channel to the first side of the first sorting channel.

In some implementations, the set of magnetic permeability particles include soft magnetic iron particles. In some embodiments, the permeability channel includes an array of pillar structures sized to stabilize the set of magnetic permeability particles within the permeability channel. In some implementations, the magnetized target entities include white blood cells labelled with magnetic beads.

In some implementations, the device also includes a second magnet placed above the substrate. In such implementations, North poles of each of the magnet and the second magnet are facing an upward direction.

In a third general aspect, the disclosure features microfluidic devices that include a microfluidic channel including an array of islands. The array of islands is arranged in one or more rows that extend along a longitudinal direction in the microfluidic channel. Each island has a width between 150 and 200 μm, a length between 200 and 800 μm, and a height between 100 and 200 μm. Each island in a row is spaced apart from an adjacent island in the row to form a siphoning channel. The array of islands is configured and arranged to shift portions of fluid through the siphoning channel between adjacent islands within a row.

In a fourth general aspect, the disclosure provides methods of concentrating and extracting particles from a sample fluid as described herein. The methods include providing the sample fluid to a first particle enrichment module of a microfluidic device as described herein. The first particle enrichment module includes a first microfluidic channel having a sample inlet at a first end of the first microfluidic channel. The microfluidic channel is configured to shift particles above a specific size to a first product outlet at a second end of the first microfluidic channel. The sample fluid is provided to the first particle enrichment module under conditions such that particle-free portions of the sample fluid are shifted through the siphoning channel between adjacent islands in a row, and an inertial lift force causes the particles above the specific size in the sample fluid to cross streamlines and transfer into a first portion of the fluid sample.

The methods also include providing a buffer fluid to a particle separation module of the microfluidic devices as described herein. The particle separation module includes a second array of islands in a second microfluidic channel, a buffer inlet in fluid communication with a first end of the second microfluidic channel, and a product inlet in fluid communication with the first product outlet and the first end of the second microfluidic channel. The second array of islands is arranged in one or more rows that extend along a longitudinal direction a corresponding microfluidic channel. Each island in a row is spaced apart from an adjacent island in the row to form a siphoning channel. The second array of islands is configured and arranged to shift portions of fluid through the siphoning channel between adjacent islands within a row to a waste outlet, and to shift particles above a specific size into a buffer flowing in the second microfluidic channel and to a second product outlet.

The methods also include passing, from the first particle enrichment module, the first portion of the fluid sample containing the transferred particles, to the particle separation module as described herein. The first portion of the sample fluid and the buffer fluid are each provided to the particle separation module under conditions such that particle-free portions of the first portion of the sample fluid and the buffer fluid are shifted through the siphoning channel between adjacent islands in a row, an inertial lift force causes the particles in the sample fluid to cross streamlines and transfer into a collection channel of the microfluidic device.

In a fifth general aspect, the disclosure features methods of concentrating and extracting particles from a sample fluid as described herein. The methods include providing the sample fluid to a sorting channel of a microfluidic device as described herein and providing a fluid containing a first set of magnetic permeability particles to a permeability channel. The permeability channel is adjacent to a first side of the sorting channel. The methods also include applying, using a magnet placed underneath the sorting channel, a deflecting magnetic field that causes a subset of magnetized target entities in the sorting channel to be deflected away from the first side of the sorting channel. The first set of magnetic permeability particles are configured to adjust the deflecting magnetic field generated by the magnet.

One or more implementations of the methods can include the following optional features. In some implementations, the method also includes providing a buffer fluid to the sorting channel of the microfluidic device at a flow rate such that flow of the buffer fluid in the sorting channel maintains particle flow of the sample fluid to be directed towards the first side of the sorting channel.

In some implementations, the method also includes providing a second fluid containing a second set of magnetic permeability particles to a second permeability channel. The second permeability channel is adjacent to a second side of the sorting channel opposite to the first side of the sorting channel. The method also includes applying, using the magnet, the deflecting magnetic field to cause a second subset of magnetized target entities in the sorting channel to be deflected towards a second side of the sorting channel.

In some implementations, the methods include passing, from the sorting channel and to a second sorting channel, a portion of the sample fluid flowing out of the sorting channel from the first side of the sorting channel and the second side of the first sorting channel. The second sorting channel is adjacent to a side of the permeability channel that is opposite to a side of the permeability channel that is adjacent to the sorting channel.

In some implementations, the supple fluid includes a leukapheresis sample.

In yet another general aspect, the disclosure provides microfluidic devices for size-based sell sorting as described herein. The microfluidic devices include a particle enrichment module that includes a first microfluidic channel having a sample inlet at a first end of the first microfluidic channel and a first product outlet at a second end of the first microfluidic channel. The microfluidic devices also include a particle separation module that includes an array of islands in a second microfluidic channel having a sample inlet at a first end of the second microfluidic channel, a buffer inlet in fluid communication with the first end of the second microfluidic channel, and a product inlet in fluid communication with the first product outlet and the first end of the second microfluidic channel. Each island is spaced apart from an adjacent island in the array of islands to form a siphoning channel. The array of islands is configured and arranged to shift portions of fluid through the siphoning channel between adjacent islands to a waste outlet at a second end of the second microfluidic channel, and to shift particles above a specific size to a first product outlet at the second end of the second microfluidic channel. The first particle enrichment module and the particle separation module are serially arranged such that a sample fluid flows from the first microfluidic channel to the second microfluidic channel.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1A is a schematic of a microfluidic device including multiple inertial separation arrays (NISAs) for size-based CTC sorting.

FIG. 1B is a schematic of a size-based sorting technique that combines inertial focusing and repetitive flow-shifting used by the microfluidic device depicted in FIG. 1C.

FIG. 1C is a schematic of a size-based CTC sorting microfluidic device.

FIG. 1D is a schematic of a NISA module construction for the microfluidic devices depicted in FIGS. 1A and 1B.

FIG. 2A is a schematic of a permeability-enhanced ultra-high-throughput magnetic sorting device.

FIG. 2B is a schematic showing a microfluidic approach for isolation of untouched CTCs from leukapheresis products using the microfluidic device depicted in FIG. 2A.

FIG. 2C is a schematic of a two-staged magnetic sorting technique used by the microfluidic device depicted in FIG. 2A.

FIG. 2D is a schematic showing high permeability of the microfluidic device depicted in FIG. 2A. The top portion of FIG. 2D illustrates using soft-magnetic iron particles prevented from escaping with flow by the filter pillars. The bottom portion of FIG. 2D illustrates an example of the microfluidic device depicted in FIG. 2A.

FIGS. 3A-G includes graphs of experimental results of a NISA design in which size sorting occurs at the break between islands at the end of each channel.

FIG. 4 is a graph of experimental results showing depletion performance of a size-based CTC sorting device with respect to platelets, red blood cells, and white blood cells.

FIGS. 5A and 5B are a pair of graphs of experimental results showing retention of CTCC structures in size-based CTC sorting devices, and related microscope images of the corresponding devices.

FIG. 6 is a graph of experimental results showing rare cell isolation of spiked CTCCs.

FIGS. 7A-7C are a graph and two microscope images that show experimental results of measured growth rates of spiked MGH-BRx-142 cells after isolation with a microfluidic device. FIG. 7A is a graph of measured growth rates, and FIGS. 7B and 7C are microscope images of cell cultures of unprocessed cells (FIG. 7B—control) and spiked CTC cells (FIG. 7C).

FIG. 8 is a graph of experimental results of flow control reliability of the microfluidic device depicted in FIG. 1A.

FIG. 9 is a graph of experimental results of blood throughput (left), total blood processed (middle), and product volume per nominal tube of blood (right) measured using the microfluidic device depicted in FIG. 1A.

FIGS. 10A and 10B are a pair of microscope fluorescence images collected when using a single array to process whole blood. FIG. 10A shows spiked CTCC concentrate in whole blood, and FIG. 10B shows sample volume with 20 μm beads.

FIG. 11A is a graph that shows experimental results of isolation of putative CTCCs from melanoma patients. In particular, FIG. 11A is a graph showing apparent cell size measured for CTCCs isolated using two microfluidic devices (“NISA-XL” and “CTC-iChip”). FIG. 11B is a series of microscope fluorescent images for different types of cell clusters (merged cells, DNA, CTCs, WBCs) of different diameters isolated using the two microfluidic devices described herein.

FIGS. 12A-12C are a graph (12A) and a pair of schematic diagrams (12B and 12C) relating to use of magnetic permeability strips between magnets in the microfluidic device depicted in FIG. 2A. FIG. 12A shows an enhancement in generating a field gradient by using permalloy strips. FIG. 12B shows a configuration of the microfluidic device in which a magnetic permeability strip is placed between magnets. FIG. 12C shows a configuration of the microfluidic device in which no magnetic permeability strip is used.

FIG. 13 shows fluorescence streak images of white blood cells at various positions of the microfluidic device depicted in FIG. 2A.

FIGS. 14A-14D are schematics of arrangements of magnets and high-permeability channels (14A, 14C) and contour plots of corresponding magnetic field intensities (14B, 14D). FIG. 14A shows an example of an arrangement of magnets and high permeability channels in the microfluidic device depicted in FIG. 2A. FIG. 14B is a contour plot of the magnetic field intensity associated with the arrangement depicted in FIG. 14A. FIG. 14C shows an example of an arrangement of magnets and high permeability channels another magnetic sorting device. FIG. 14D is a contour plot of the magnetic field intensity associated with the arrangement depicted in FIG.

FIG. 14E is a graph comparing the magnetic field gradients measured in the microfluidic device depicted in FIG. 2A and another magnetic sorting device.

FIG. 14F is a graph comparing the lateral deflection velocities of magnetic beads measured using particle-tracking velocimetry.

FIG. 14G is a vector plot of the gradient of the magnitude of the magnetic field in a sorting channel of the microfluidic device depicted in FIG. 2A.

FIG. 14H is a graph showing lateral deflection measured for beads and cells in the first stage sorting channel (212) of the microfluidic device depicted in FIG. 2A.

FIG. 14I is a graph showing lateral deflection measured for beads and cells in the second stage sorting channel (224) of the microfluidic device depicted in FIG. 2A.

FIG. 15A includes graphs comparing depletion data for processed Leukopak® samples (left), leukapheresis samples, and product isolated using a microfluidic device. The left-middle graph compares depletion data for red blood cells between the leukapheresis samples and the isolated product. The right-middle graph compares depletion data for white blood cells between the leukapheresis samples and the isolated product. The right graph compares depletion data for platelets cells between the leukapheresis samples and the isolated product. Cell numbers in full leukapheresis samples are depicted in black and mimic samples are depicted in gray.

FIG. 15B is a graph of the CTC isolation yield measured based on the depletion data depicted in FIG. 15A.

FIG. 15C includes immunofluorescence images of isolated spiked CTCs stained using EpCAM (green) and DAPI (blue).

FIG. 15D includes a graph comparing in vitro growth of isolated CTCs measured in an isolated product and a control sample.

FIG. 15E is a graph expressing breast lineage markers from spiked cells after CTC enrichment as measured by ddPCR analysis.

FIG. 16 is a graph showing the effect of blood volume and CTC occurrences on the likelihood of sampling greater than 50 CTCs.

FIG. 17 includes graphs of viSNE clustering views of two markers, CD45RA (left) and CD3 (right) from markers used for profiling of leukocytes present in isolated product.

FIG. 18A is a schematic showing the principle of separation in a non-equilibrium insertional separation array chip. FIG. 18B is an image of a portion of an non-equilibrium insertional separation array chip.

FIG. 18C is a photograph of a system with running platform for separation assays.

FIG. 19A is a schematic of a cell contraction module that can be incorporated into the microfluidic device depicted in FIG. 2A.

FIG. 19B includes schematic views of inertial focusing of a dilute cell suspension through asymmetric serpentine units of the cell contraction module depicted in FIG. 19A.

FIG. 20 includes schematics showing the product-to-waste cutoff in the first stage sorting channel (212) of the microfluidic device depicted in FIG. 2A (top) and the product-to-waste cutoff in the second stage sorting channel (224) of the microfluidic device depicted in FIG. 2A (bottom).

FIGS. 21A and 21B are graphs showing the variations in magnetic field gradients measured in a sorting channel of the microfluidic device depicted in FIG. 2A. FIG. 21A is a graph showing variations in the x-component of a magnetic field gradient. FIG. 21B is a graph comparing the x-component and the y-component of the magnetic field gradient.

FIG. 21C is a schematic diagram illustrating forces on cells undergoing magnetophoretic deflection toward the center of a sorting channel.

FIG. 22A is a schematic showing a perspective view of the microfluidic device depicted in FIG. 2A.

FIGS. 22B and 22C are schematics showing a manifold that can be used hold the microfluidic device depicted in FIG. 2. FIG. 22B shows the top and bottom portions of the manifold. FIG. 22C shows an assembled configuration of the manifold.

FIG. 23 shows graphs for CTC yield data for three cell lines when only 5 cells were spiked.

FIG. 24 includes graphs of assays performed using cell lines MGH-BRx-14, MDA-MB-231, and LNCaP.

FIG. 25A shows different arrangements of magnets that enable alignment-free magnetic field focusing using the microfluidic device depicted in FIG. 2A.

FIG. 25B is a graph comparing the magnetic field gradients measured using different arrangements of magnets.

FIG. 25C is schematic of a microfluidic device that can be used with arrangements of magnets that enable alignment-free magnetic fields.

FIG. 25D are photographs of microfluidic channels with different arrangements of magnets.

Like reference symbols in the various drawings indicate like elements.

### DETAILED DESCRIPTION

Systems and techniques are described herein for isolating target entities, such as intact circulating tumor cells (CTCs) and circulating tumor cell clusters (CTCCs), from large volumes (e.g., 100-200 mL) of samples (e.g., whole blood or leukapheresis samples) with high purity and low shear. The new high-throughput microfluidic systems are capable of isolating CTCs and CTCCs with high yield, extremely low carryover of blood cells, and requiring minimal or no dilution of blood, and thereby providing an ability to conduct CTCC collection and analysis, as well as apheresis.

For reliable cell sorting, specifically for sorting of rare cells, it is important to isolate cells in greater numbers from large volumes. Processing up to 100-200 mL whole blood samples or 65 ml Leukopaks® generated from human blood in an apheresis system are ideal start points for CTC isolation. These samples can lead to isolation of a larger number of viable CTCs, which will not only increase the likelihood of successful CTC culture, but will also enable early stage cancer diagnostics. As discussed herein, in some implementations, a size-based microfluidic debulking device is used to isolate CTCCs from whole blood. In other implementations, a permeability-enhanced ultra-high-throughput magnetic sorting device can be used alternatively or in combination with the size-based sorting microfluidic device to recover CTCs or CTCCs from large volume samples.

### A. Systems Overview

As described herein, any one, or any combination of two or three of the following technologies can be used to isolate CTCs and CTCCs from blood samples, e.g., whole or minimally diluted blood samples. First, CTCCs can be captured by positive selection (e.g., by EpCAM antibody binding), though not all CTCs express membrane-bound EpCAM, for example, after epithelial-mesenchymal transition or in non-epithelial melanoma. Second, WBCs can be removed from CTCCs by targeting CD45. This negative selection strategy can be employed by a microfluidic cluster sorter, as discussed in detail below. Applying this strategy, bead-labeled leukocytes are removed by magnetic activated cell sorting (MACS), after RBC size-sorting upstream. Some CTCCs may be lost in negative selection if they associate with WBCs. A third approach involves separating CTCCs from blood by their larger size or different shape (referred throughout as size-based filtration of CTCCs). Single CTCs often overlap with WBCs in size, yet even two-cell clusters are sufficiently larger than WBCs to allow a clean separation, and when sorted by their long-axis, this difference can be maximized.

CTCCs have been found to be 50-100 times more metastatic than single CTCs, which makes them a particularly relevant target for liquid biopsy. CTCCs are exceedingly rare in blood and can break up if exposed to too much fluid shear, splitting their unique bioinformatic signatures.

a. Size-Based Microfluidic Debulking Devices

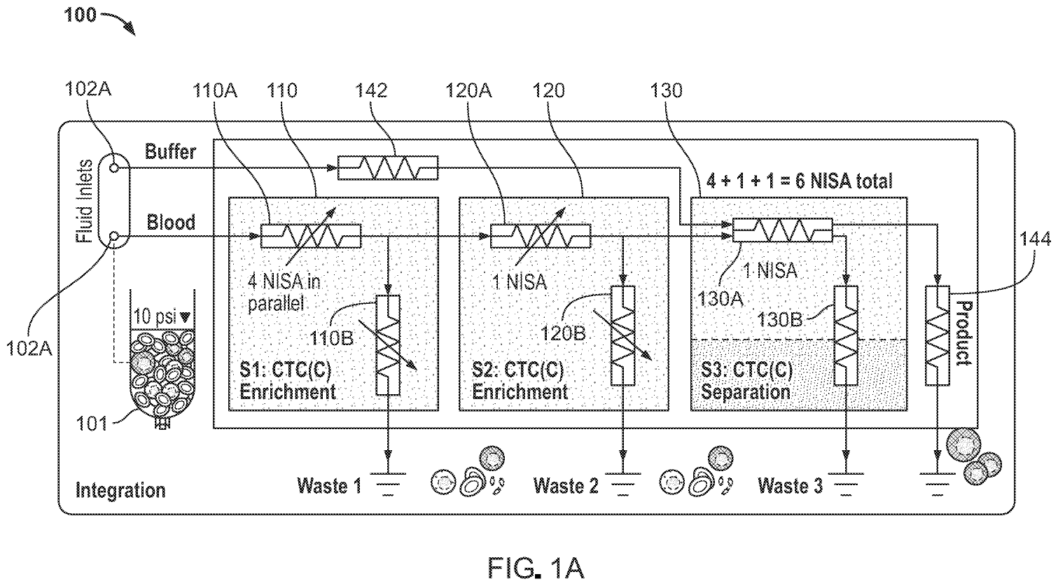

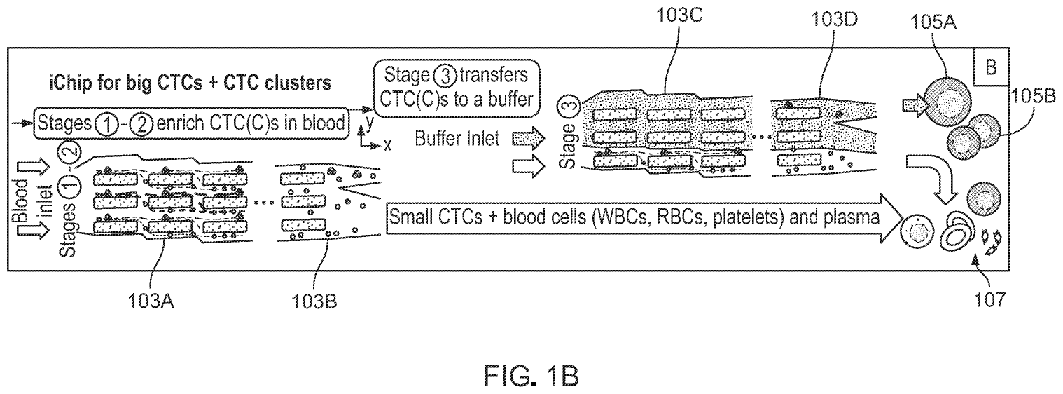

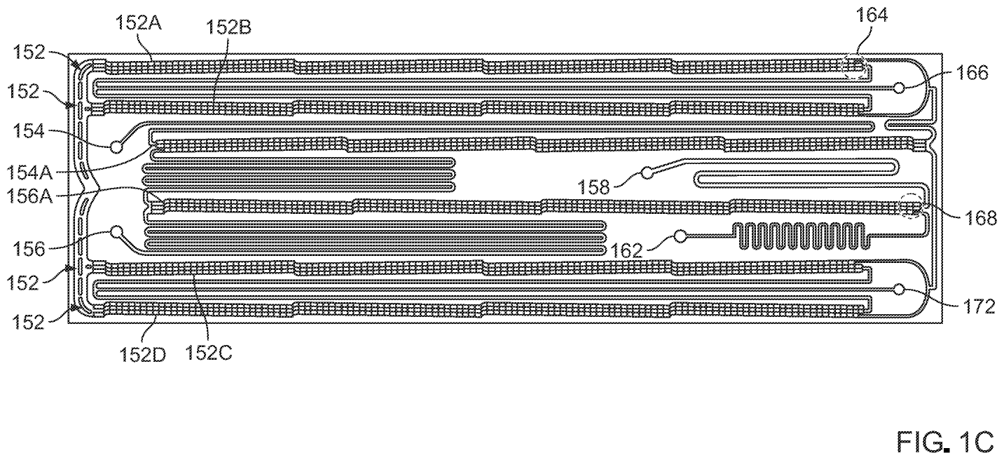

FIG. 1A is a schematic of a microfluidic device 100 for size-based sorting for CTCC isolation. The microfluidic device 100 enables separation of CTCCs from large sample volumes (e.g., 100 to 200 mL) with the application of low shear stress while requiring minimal or no blood dilution. To accomplish this, the microfluidic device 100 includes microfluidic channels with multiple non-equilibrium inertial separation array (NISA) modules that improve blood debulking by combining wall lift forces with array fluid-shifting principles. The microfluidic channels can have channel widths between approximately 80 μm to 120 μm (e.g., a 90 to 110, e.g., 100 μm channel width), channel lengths between 200 μm and 800 μm (e.g., 300 to 600, e.g., 400 to 500, e.g., 450 μm channel length), and channel heights between 100 μm and 200 μm (e.g., 125 to 175, e.g., 140 to 160, e.g., 150, 151, 152, 153, 154, 155, 156, 157, 158, or 159 μm channel height).

The NISA modules enable inertial lift to push cells away from channel walls in a size-dependent manner (as discussed in detail in reference to FIG. 1D). NISA modules incorporate rectangular structures (referred throughout as “islands”) between which inertial focusing occurs in relatively wider channels (depicted in FIG. 1B). The islands can be constructed according to a ratio of island length to the island width between 1:1 (i.e., equivalent island length and island width) and 10:1 (i.e., island length being ten times greater than island width). In some instances, the islands are constructed to have a 4:1 length-to-width ratio.

In various implementations, channel fluidic resistances of each of the NISA modules that are in parallel can be used to determine the fluid shift fraction. For example, four NISA modules can be configured in parallel in a single array, including two NISA modules with 100 μm siphoning channels between islands, one product NISA module of variable island widths, and one waste NISA module of variable island widths. In this example, as waste channel width increases from one trio of islands to the next (in the flow direction), product channel width decreases. Some of the flow in the product lane is transferred ultimately to the waste channel. The islands could actually be different width and length and the same fluid shift could be achieved (with a small modification to account for the increased siphon channel resistances).

In some implementations, at the end of each channel segment between islands, about 3-4% of sample flow is siphoned through a narrow gap between one island and its adjacent island. Because larger cells remain in the channels and smaller cells are siphoned, this progressively moves large cells to an upper product lane, leaving small cells to exit below. The wall lift force is strongly dependent on the mean diameter of cells. Thus, WBCs, RBCs and platelets are siphoned away while the larger clusters of cells are not.

As discussed herein, the NISA design of the microfluidic device 100 provides improvements relating to cell isolation and removal from a sample fluid by increasing throughput and purity. For example, one of the main causes of lower product purity in NISA modules is two roller vortices that form at the trailing edge of each rectangular post as flow speed increases. As a result, small particles (like RBCs and platelets) sometimes enter the product lane despite falling below the predicted size cutoff. To improve the product, siphon channels can be made narrower and longer, which results in significant reductions in carryover of RBCs and platelets. Secondly, for improving the throughput, a multistage sorting architecture with NISA technology can be used to enrich rare CTCs and nucleated cells (e.g., WBCs) in whole blood in two stages. In the final stage, a buffer co-flow is introduced to enable separation of WBCs and CTCs in a clean buffer. This allows high purity isolation of WBCs and CTCs from platelets, plasma and RBCs. This architecture also provides achieve significantly higher throughput while using fewer NISA modules. The architecture can also be applied to sorting nucleated cells from blood (i.e. a lower size cutoff), though the descriptions herein are focused on CTC and CTCC isolation. This architecture can be used to increase sample throughput relative to an architecture that employs a single pair of co-flow exchangers.

Larger NISA constructions can sometimes complicate parallelization and take approximately four times as much space on-chip. In some instances, sample throughput can also be limited by low injection of 15-18%. As discussed in reference to FIGS. 1A and 1B, five upstream NISA modules (in particle enrichment modules 110 and 120) are inserted without a buffer co-flow (particle enrichment modules 110 and 120). Together, these NISA modules concentrate CTCCs within a sample by approximately 10 times after two stages with 100% sample injection (4 NISA modules in stage 1 and one NISA module in stage 2). This technique also removes about 90% of the blood. One co-flow stage separates the CTCCs as they transit to the product outflow tubing (in stage 130). In the 100% injection section of the microfluidic device, the sample with blood cells and smaller CTCs (e.g., green streaks) leaves through waste outlets 166, 172, and 154, while large CTCs and CTCCs exit in the product lane of stages 1 and 2. Large cells and CTCCs can be seen exiting the particle separation module 130 alongside waste outlet 158 (remaining sample plus co-flow buffer). Notably, by increasing sample injection from 15% to 100% in particle enrichment modules 110 and 120, 15 mL of whole blood can be processed in 30 minutes from six NISA modules on a single standard slide-sized device (depicted in FIG. 1C) with relatively low shear stress, without even a filter to remove debris.

Referring to FIG. 1A, the microfluidic device 100 includes six NISA modules 110A, 110B, 120A, 120B, 130A, and 130B that are arranged to provide three interconnected sorting stages 110, 120, and 130. For example, NISA modules in each individual stage (e.g., NISA modules 110A and 110B in particle enrichment module 110) are arranged in serial, whereas one NISA module in each stage is arranged in parallel to a NISA module in a different stage (e.g., NISA module 110B in particle enrichment module 110 is arranged in parallel to NISA module 120B in particle enrichment module 120). As discussed below, this architecture enables iterative sorting to reduce loss of CTCCs in sample fluid processed using the microfluidic device 100.

To reduce blood processing time, CTCCs are first concentrated by about ten times in the blood without a co-flow. Then, CTCCs are separated into clean buffer by a final stage of NISA (15% injection of sample alongside buffer). After tuning flow rate for the desired size cutoff and establishing high yield isolation of intact CTCCs by high speed video, performance can be reported for runs of 15 mL whole blood that take, for example, approximately 30 minutes each. Depletion levels are high at 4.2 for WBCs, 5.5 for RBCs, and 4.9 for platelets (in base-10 logs). In addition, five 2-cell and three 3-cell CTCCs were manually spiked into multiple 15-mL blood aliquots and detected in product wells.

NISA module 110A is a 100% injection device (i.e. fluid received by the NISA module is an undiluted sample fluid). NISA module 110A includes four NISAs arranged in parallel and feed one NISA included on NISA module 120A (which is also a 100% injection device). NISA module 120A then feeds one NISA included in NISA module 130A. The NISA included in NISA module 130A also receives a co-flowing feed of a buffer sample from NISA module 142. The output of NISA module 130A is separated between NISA module 144 (which receives a product volume with isolated CTCs and/or CTCCs) and NISA module 130B (which receives waste volume in a similar manner as NISA modules 110B and 120B. Input (blood and buffer), output (wastes 1-3 and product), and internal flow splits (stages 1-3 product, stage 3 injection) are controlled by a network of channel resistances.

The microfluidic device 100 can be created with polydimethylsiloxane (PDMS) soft lithography techniques. Su8-100 was coated onto silicon wafers by spin-coating at 2000 rpm for 30 seconds, to a thickness of about 156 μm. UV photolithography (365 nm) was then used to create a single layer of microchannels on coated silicon wafers. PDMS (Sylgard 184) was then poured onto the resulting channel molds at a ratio of 9:1 base to cross-linker. After cutting cured PDMS from the mold and dicing devices, biopsy punches were used to create holes for press-fit tubing to connect sample inlet, buffer inlet 156, waste outlets 166 and 172, waste outlet 154, waste outlet 158, and product outlets 162. Channels were enclosed by permanently bonding each 3×1 in² device to a glass microscope slide in an oxygen plasma oven. Devices were then baked immediately at 70° C. for 10 minutes and later at 150° C. for 3 hours with gradual ramps. This last bake helps to increase the elastic modulus of the PDMS, such that microchannel dimensions are not enlarged too much in upstream NISA modules.

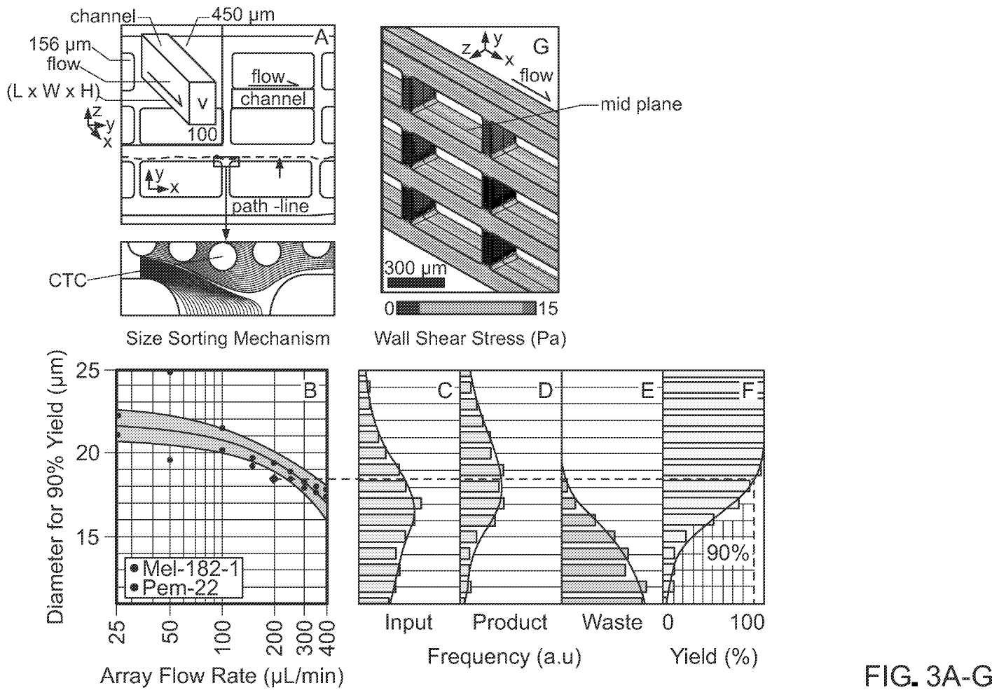

FIG. 1B is a schematic of a size-based sorting technique that combines inertial focusing and repetitive flow-shifting used by the microfluidic device 100 depicted in FIG. 1A, as discussed above. In a first stage 103A and a second stage 103B, a 100% sample is injected into a microfluidic channel and flowed through NISAs to enrich concentrate CTCCs in the sample (referred to as enrichment). In a third stage 103C, the sample is co-flowed with buffer so that non-target cells 107 (e.g., small CTCs, RBCs, WBCs, platelets) progressively migrate to the bottom of the channel through siphoning gaps. As depicted in 103D, target cells (e.g., large CTCs 105A and CTCCs 105B) migrate away from the bottom of the channel and towards a product output. The fluidic resistance within the channel can be adjusted based on dimensions of the channel and the islands that comprise a NISA, as discussed in greater detail with respect to FIG. 3.

FIG. 1C is a schematic of a size-based CTC sorting microfluidic chip 150. A blood sample is introduced through four inlet ports 152 that are each in fluidic communication with a respective NISA module. NISA modules 152A, 152B, 152C, and 152D are arranged in parallel with each other. Sample flow through the NISA modules 152A-D represents the first stage (depicted in particle enrichment module 110 in FIG. 1B) of the iterative size-based cell sorting technique.

A portion of the sample fluid flowing through NISA modules 152A and 152B that includes non-target cells exits through a waste outlet 166. In a similar way, the portion of the sample fluid flowing through NISA modules 152C and 152D that includes non-target cells exits through a waste outlet 172. The other portions of the sample fluid proceed to the second stage of the size-based cell sorting technique (shown as particle enrichment module 120 in FIG. 1B).

In the second stage, sample fluid is flowed through NISA module 154A. A portion of the sample fluid that includes non-target cells exits through waste outlet 154. The remaining portion of the sample fluid proceeds to the third stage of the size-based cell sorting technique.

In the third stage, sample fluid is co-flowed in NISA module 156A with buffer introduced through inlet port 156. A waste portion of the buffer/sample fluid flowing through the NISA module 156A exits through waste outlet 158. The remaining portion (which includes target cells) exits the microfluidic device 150 through product outlet 162.

In some implementations, 15-20% of a 400 mL blood sample (e.g., 1:1 diluted blood) is injected into microfluidic device 150 to isolate leukocytes in approximately three hours. This technique can also be used to remove approximately greater than 99.99% of RBCs, and thereby sort approximately 300 million cells per second in 104 parallel arrays. The applied wall shear stress using this technique was measured to be approximately 100 Pa, and channel width was around 50 μm.

Design parameters of NISA modules (e.g., channel size) and flow rate applied can be used to reduce sample residence time in the microfluidic device 150 and thereby minimize sample clogging. In some instances, fluid forces are leveraged to shift CTCCs and RBCs away from channel walls, thereby facilitating separation in larger channels. Focusing techniques (e.g., inertial focusing, acoustic focusing) can also further reduce clogging. For example, inertial focusing can be used to trap large cells in micro-vortex traps, processing blood at, for example, 22.5 mL/hour, though wall shear stress may be too high in narrow intervening channels to keep CTCCs intact (up to 1000 Pa, 200× peak arterial). Inertial focusing in spirals has lower wall shear stress and can process blood at, for example, about 3 mL/hour with dilution. Spirals require Dean flow to drag cells laterally, resulting in mixing and carryover to the product of unfocused material (e.g. RBC fragments, platelets, plasma). Carryover can be reduced by lysing the RBCs. This technique precludes apheresis and adds stress to clusters. Acoustic focusing can also process blood at low shear, though purity is lower in comparison.

FIG. 1D is a schematic of a NISA module construction for the microfluidic devices depicted in FIGS. 1A and 1B. As shown, a NISA module 182 includes an array of islands 184 that are arranged to create separation channels (between rows of islands) and siphoning channels 186C (between adjacent islands, such as between points 186A and 186B. The siphoning channel dimensions can be configured to enable rapid, gentle, size-sorting of CTCCs from blood. For example, the width of the separation channel 182 can be between 50 μm and 200 μm (e.g., 100 μm width), and the height can be between 100 μm and 400 μm (e.g., 150, 151, 152, 153, 154, 155, 156, 157, 158, or 159 μm height). Additionally, array parameters, such as island length (e.g., 200-800 μm, such as 450 μm), flow shift percentage (e.g., between 2-10%, such as between 3-4%) can also be specified so that size cutoff rises to between the largest leukocytes and smallest CTCCs (or large single CTCs) to approximately 15 μm. In this manner, wall shear stress falls by a factor of seven to less than 15 Pa in six-fold larger cross-section channels to enable larger CTCCs to pass.

The geometry of siphon channels 186C depicted in FIG. 1D results in vortex formation at points 186A and 186B. This is caused by fluid inertia as flow rate (and Reynolds number) is increased. In this configuration, the vortices are constrained, which reduces mixing from one point to the other. The dimensions of the siphon channels 186C also reduce the mixing in the final inertial exchanger that is used to introduce a co-flowing buffer (depicted in FIG. 1B).

Importantly, because the product lane remains clean of RBCs (and any particles below the size cutoff), the CTCCs exit in a product flow of high purity. Interestingly, this is a per-array flow rate about ten times higher than prior iterations of NISA modules used for CTCC isolation. Despite the dramatically higher Reynolds number, mixing is tamed enough to keep the product lane clean. The size cutoff can be set between size of RBCs and WBCs (approximately 7 μm) and the size of large CTCs and two-cell CTCCS (approximately 18-20 μm). In some implementations, the NISA modules are constructed such that a channel width is between 50 and 100 μm, channel length is between 200-450 μm, a depth of approximately 156 μm.

The dimensions of the siphoning channels discussed above can reduce the impact of strong inertia-driven vortices on the trailing edges of the islands mix cells from sample to buffer, which often spoils CTC and CTCC separation. For example, by narrowing and lengthening the siphoning channels, vortex formation at points 186A and 186B can be physically separated and thereby prevent RBCs from mixing from one vortex to another. This aspect can improve purity of separation due to fewer RBCs being present in the product of the final separation stage.

b. Permeability-Enhanced Ultra-High Throughput Magnetic Sorting Devices

Microfluidic immunomagnetic sorting can allow highly customizable sorting of rare cells. However, many other magnetic sorters can only process up to 10-20 ml of blood sample in an hour since handling a larger sample volume (e.g., greater than 100 mL) requires development of a microfluidic device that is capable of sorting cells at a high yield without clogging.

A standard blood tube for diagnostic analysis contains 10 mL of peripheral blood, from which approximately 1 to 50 CTCs may be isolated, depending on tumor type and stage of disease. While collecting large numbers of blood tubes from patients with cancer is prohibitive, leukapheresis is a well-tolerated routine clinical procedure, in which large volumes of blood (˜5 L) are processed, with centrifugal enrichment of peripheral blood mono-nuclear cells (PBMCs) into a Leukopak® of approximately 65 mL volume during an hour-long procedure. The remaining constituents of the blood, including plasma, RBCs and most neutrophils, are returned to the patient. CTCs by virtue of having a similar density as mono-nuclear cells (1050-1080 kg/m³) are enriched in a leukapheresis product.

While leukapheresis allows for initial cell density-based sorting of entire blood volumes, current CTC isolation technologies can only process up to 200 million mono-nuclear cells or about 3 to 5% of a Leukopak®, which often limits the benefit of processing leukapheresis products. The microfluidic devices described herein can process an entire leukapheresis volume of 65 mL and is capable of recovering thousands of untagged viable CTCs by depleting RBCs, platelets, and WBCs in a tumor-agnostic manner. Inertial separation array devices can be incorporated to allow for removal of RBCs and platelets followed by a high gradient magnetic cell sorter for the depletion of WBCs. The development of this ultrahigh-throughput permeability-enhanced magnetic cell sorter enables depletion of 50 to 100-fold more WBCs than current magnetic depletion platforms and is useful to the processing of large blood volumes for CTC enrichment at an unprecedented scale.

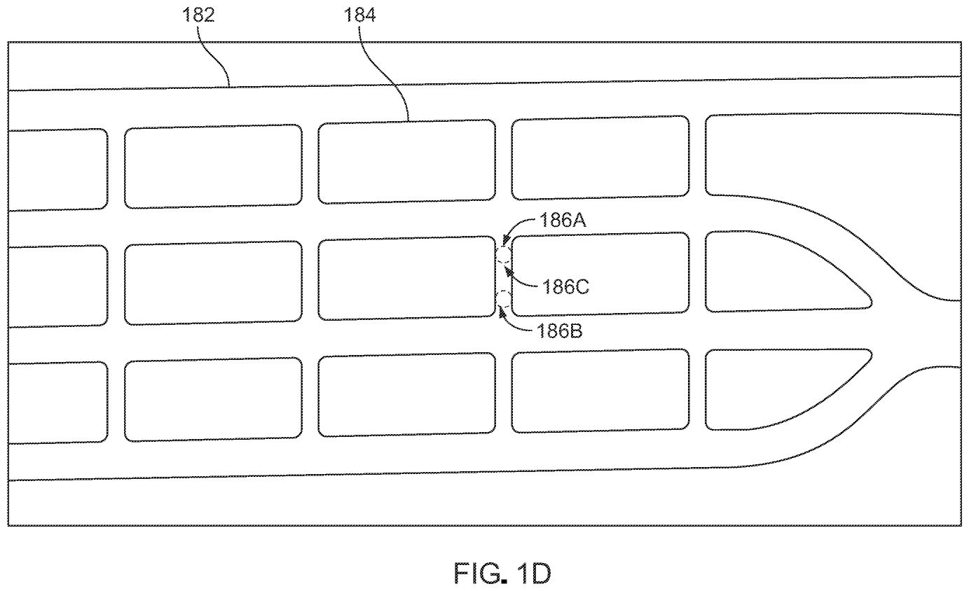

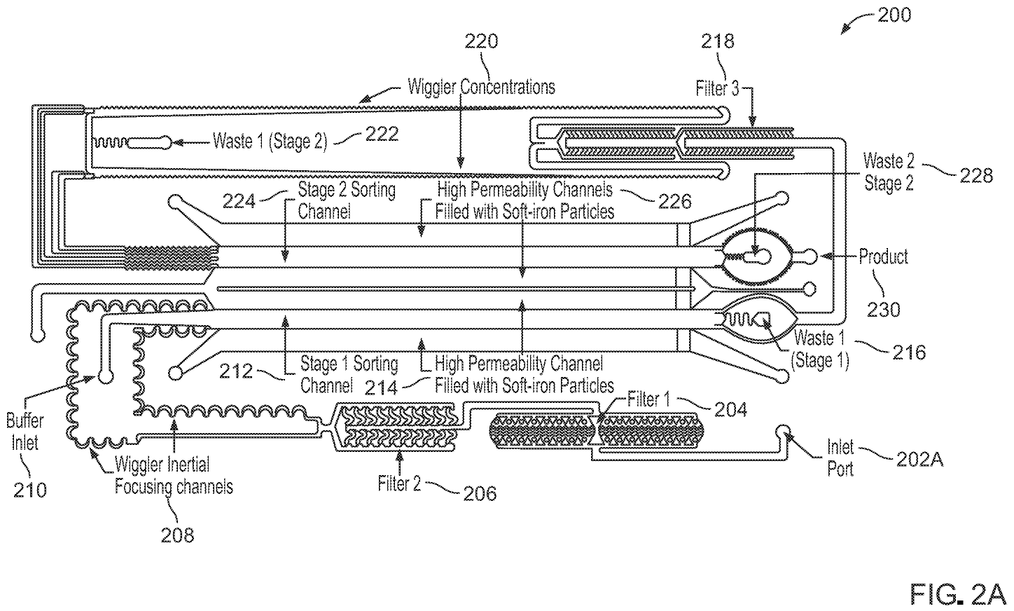

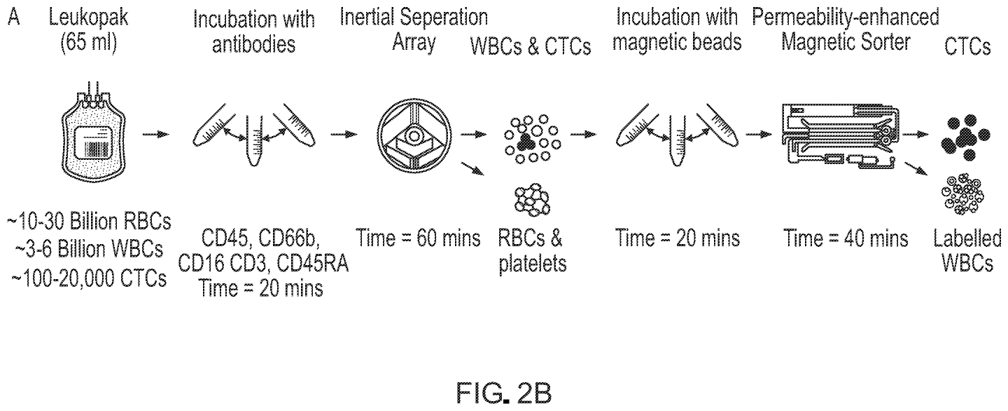

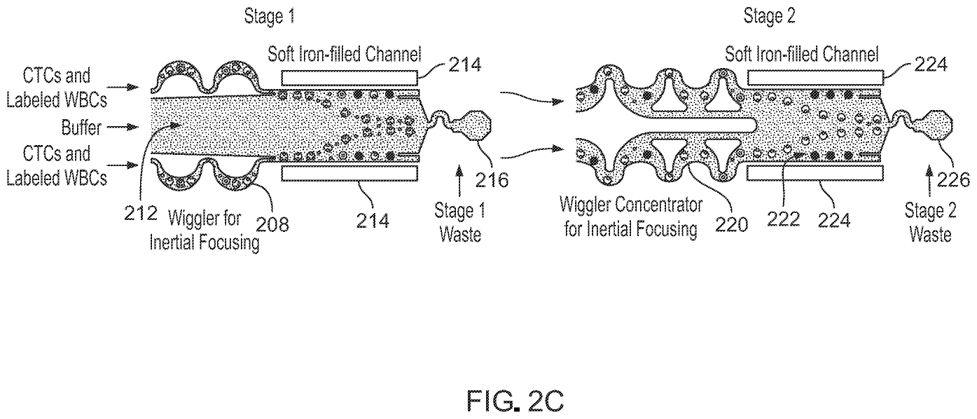

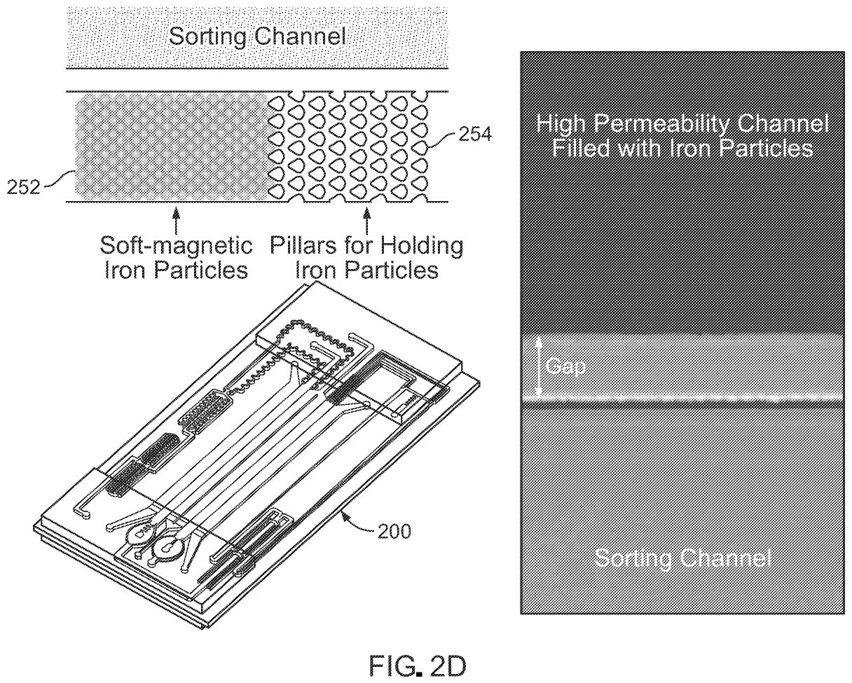

FIG. 2A is a schematic of a microfluidic device 200. The microfluidic device 200 permits isolation of target cells or cell clusters (e.g., CTCCs) through an iterative, multi-stage, e.g., two-stage, sorting process (depicted in FIG. 2C). In the first stage, a sample is initially introduced into the microfluidic device 200 through inlet port 202A. The sample proceeds through filters 204 and 206 and flowed into inertial focusing channels 208 before being introduced into a sorting channel 212. A high-gradient magnetic field is applied to the sorting channel 212 for magnetic deflection. The magnetic field is applied by introducing high permeability material in a high permeability channel 214 adjacent to sorting channel 212.

As depicted in FIG. 2D, high permeability channel 214 is packed with micron diameter between 5 μm and 500 μm, e.g., 10, 20, 30, 40, 50, 60, 70, 80, 90, 100, or greater than 200 μm, and preferably 30 μm. Magnetic permeability particles 252 dispersed in a generally inert carrier fluid, such as water or ethanol. The magnetic iron particles 252 can be any type of particles that are capable of focusing magnetic fields in surrounding material, such as soft iron particles. The dispersed solution is then passed through pillars 254 near the end of the high permeability channel 214, which permits fluid to escape the channel, but traps particles in the channel.

High permeability channel 214 is created by filling a microfluidic channel created in the same photolithographic step used to create sorting channel. This configuration enables precise alignment of high gradient material close to the sorting channel 212 by enabling high magnetic field gradients, which is used for rapid cell sorting. A waste portion of the sample fluid (e.g., fluid containing non-target cells) that flows through the sorting channel 212 exits the microfluidic device 200 through waste outlet port 216. The remaining portion proceeds along the fluidic circuit to the second stage.

In the second stage, a portion sample fluid passes through filter 218 before flowing through wiggler concentrators 220. A waste portion of the sample fluid that passes through the wiggler concentrators 220 exits the microfluidic device 200 through waste outlet port 222. A remaining portion of the sample fluid passes is introduced into a sorting channel 224. A high-gradient magnetic field is applied to the sorting channel 224 in a similar manner as discussed above for sorting channel 212. A waste portion of the sample fluid that flows through the sorting channel 224 exits the microfluidic device 200 through waste outlet port 228. The remaining portion (which includes target cells) exits the microfluidic device 200 through product outlet 230. In some implementations, the microfluidic device 200 is configured to operate at a throughput of 100 mL/h and removes 99.95% WBCs labeled while recovering 95% of the spiked CTCs.

To perform immunomagnetic sorting, high permeability material can be introduced in vicinity of a sorting channel in which a sample fluid is flowed. Two permeability channels can be adjoined to each side of the sorting channel. Each permeability channel can then be filled with magnetic permeability particles, such as 40 μm (in diameter) soft magnetic iron particles. For example, magnetic beads can be dispersed in a reagent (e.g., water, ethanol) and magnetized fluid can be passed through the permeability channels. As depicted in FIG. 2D, the permeability channels can include filters near the end that permit reagent fluid exit the permeability channels but retain magnetic permeability particles within the permeability channels. The permeability channels can also include pillars that further stabilize the positioning of magnetic permeability particles within the permeability channels.

The permeability channels depicted in FIG. 2A can be fabricated in different shapes using soft lithography techniques. The permeability channels can also positioned at different distances relative to a sorting channel to adjust the strength of the deflective magnetic field applied to the sorting channel. As discussed herein, the introduction of a magnetic fluid (e.g., magnetic permeability particles dispersed in a reagent) into permeability channels enables magnetized target entities in the sample flow to be directed towards a side of the sorting channel.

The ultra-high throughput magnetic sorting devices described provide various advantages in isolation of CTCCs and CTCs. For example, the high capacity magnetic cell depletion techniques employed can enable sorting through an entire leukapheresis product for the presence of CTCs within three hours. Compared to other CTC isolation techniques, this can increase the number of CTCs recovered by two orders of magnitude, and it may provide a noninvasive alternative to core needle biopsies of tumors that are routinely used for cancer diagnosis and monitoring.

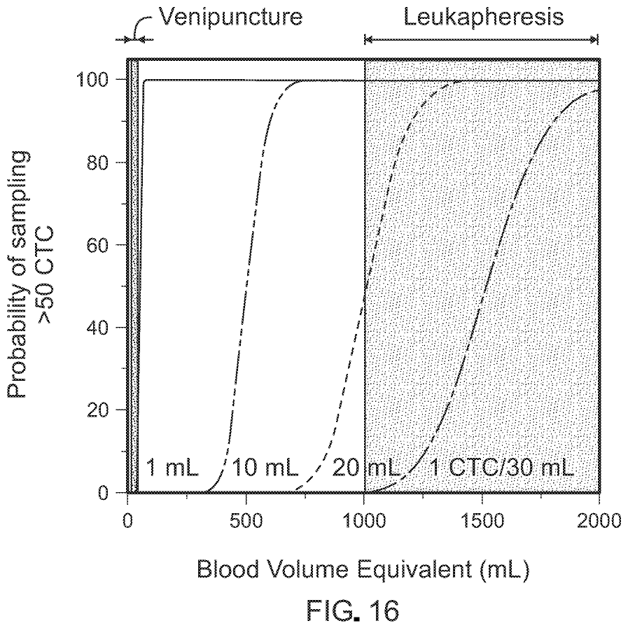

Many CTC isolation techniques are often limited by the number of CTCs present within a standard 10 mL tube of whole blood. While these have provided important insights into the process of blood-borne metastasis, incorporation of CTC-based diagnostics into clinical care often requires consistent isolation of sufficient numbers of cancer cells from the blood. One feasible avenue to capture more CTCs is to increase the volume of processed blood. For example, Poisson-distribution-based statistical modeling of random CTC sampling in blood indicates that the probability of obtaining CTCs increases predictably with the processed blood volume and CTC concentration (as depicted in FIG. 16), and a leukapheresis product, generated from ˜5 liter of blood, is the ideal starting material. However, microfluidic enrichment of cancer cells from such a large number of blood cells presents several technological challenges, particularly, using antibody-mediated negative depletion of massive numbers of WBCs to reveal untagged viable CTCs.

The magnetic sorting devices disclosed herein can efficiently process a large volume of sample fluid, e.g., 65 mL of leukapheresis product, with more than 10-fold higher concentration of WBCs and platelets compared with the peripheral blood. Operating at an ultra-high throughput, the magnetic sorting devices can achieve an 86% CTC recovery with greater than 10⁵ depletion of hematopoietic cells, without clogging, platelet activation, or release of WBC DNA nets. Recovered CTCs also have preserved viability and molecular integrity. Unlike macro cell sorting approaches such as density gradient centrifugation and bulk magnetic sorting, the magnetic sorting devices described herein are operator-independent, incur minimal rare cell loss and provide precise sorting conditions at a single cell level.

Negative depletion of hematopoietic cells also present several biological advantages compared to positive selection of CTCs. For instance, EpCAM-based positive selection of CTCs from a large background of untagged blood cells often requires less magnetic sorting, but it also limits the types of cancer cells recovered to the subset expressing high levels of this epithelial marker. In addition, the presence of bead-conjugated capturing antibodies at the tumor cell surface restricts functional viability, the quality of RNA, and accessibility for detailed imaging and morphological analysis. In contrast, negative depletion of hematopoietic cells generates unmanipulated and potentially viable CTCs.

The magnetic sorting devices also address at least several technical challenges. For example, the devices incorporate a magnetic circuit sensitive enough to deflect all unbound beads, thereby removing any possibility of bead contamination in the product. As another example, despite using high field gradients, the new devices create a clog-free flow within the microfluidic circuits of the magnetic sorting devices. During labeling, some of the WBCs disproportionately often acquire a large number of beads (e.g., >50 beads) due to high expression of antigens targeted for depletion. Under the action of traditional magnetic field design, cells with high bead loads often rapidly attach to the channel walls, forming a plaque that clogs the channel and leading to device failure. This complication is addressed in the new devices by deflecting cells toward the center of the channel in the core of the flow where no walls are present, and away from high-gradient regions. Cells with high magnetic loads are then rapidly focused at the center of the channel, which enables the new devices to process billions of cells. The symmetric force toward the center of the channel is made possible by coplanar high-permeability channels.

### B. Techniques for Isolating Rare Cells Using Size-Based Microfluidic Debulking Devices

a. Design

To settle on a NISA design, candidate devices were first generated to the appropriate length scale with the help of dynamic similarity to prior NISA chips. These designs were tested as single arrays where spiked CTC/cluster yield and blood cell depletion were the key performance metrics. Devices also needed to operate at high sample flow rate while incurring minimal wall shear stress and fit on a standard microscope slide (<7.5 cm long) to fit fabrication constraints. Ultimately, the chosen NISA design enlarged the channels from 50 to 100 μm wide and extended to 156 μm tall while increasing channel length from 200 to 450 μm (shown on left side of FIG. 3A).

As depicted in FIG. 3A, size sorting occurs at the break between islands at the end of each channel. Fluid siphoning percentage can be set to 3.6% of the main channel flow at each break between islands to achieve size sorting in the desired range. Particles that migrated beyond the red streamline remain on the +y side of the island, while smaller particles remain closer to the −y wall and are siphoned back to the next lane in the −y direction. The computational fluid dynamics model of one microfluidic device (depicted in FIG. 3G) is performed with ANSYS 13 Fluent (mesh: 3.9 million prismatic elements; midplane symmetry used to cut model in half). At 200 μL/min per array (0.8 mL/min total), peak wall shear stress is less than 15 Pa (depicted in FIG. 3G) for about 4 seconds, close to the peak shear stresses experienced in circulation (up to about 5 Pa or much higher in stenotic arteries), although cancer cells will have different sensitivities to shear stress.

b. Fluidic Control

Before introducing sample to the microfluidic device, priming is performed through a sample inlet with a priming fluid, such as 50% ethanol in deionized water, pure water (or other aqueous solutions), polyethylene glycol diacrylate gel (crosslinked after loading), low viscosity hydrocarbons (e.g., gasoline), fluorocarbons or hydrofluorocarbon oils. Viscosity of the priming fluid should not be so high that slurry cannot be pushed into the channels without damaging the microfluidic device. Ethanol and water are relatively easy to remove after loading, but the priming fluid need not be removed if the inlet and outlet ports are sealed after loading. Each port is individually addressed in the following order: waste outlets 166 and 172, waste outlet 154, buffer inlet 156, waste outlet 158, and product outlet 162. The channels and tubing are subsequently flushed with buffer (1% w/v F127 in PBS) via the buffer inlet 156 in reverse order, finishing with the sample line. Sample processing is then initiated.

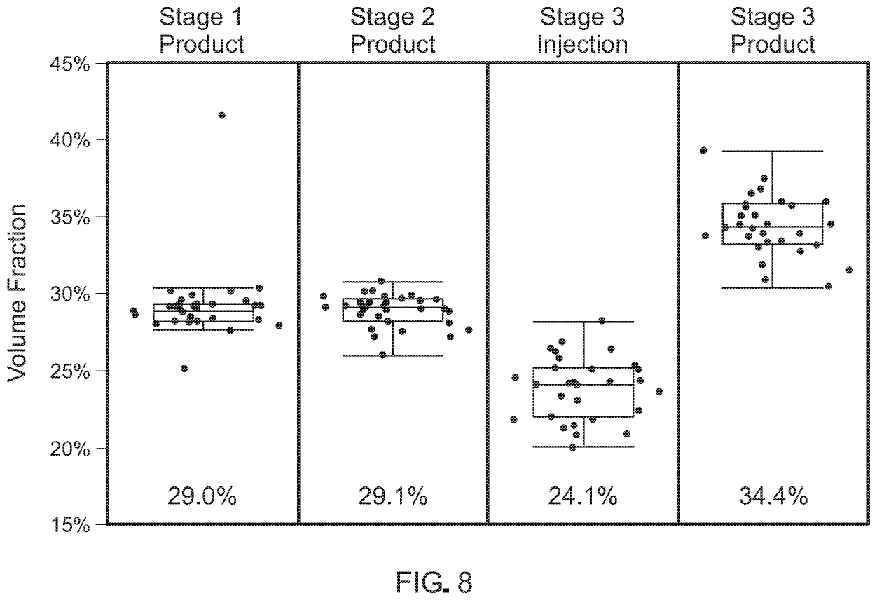

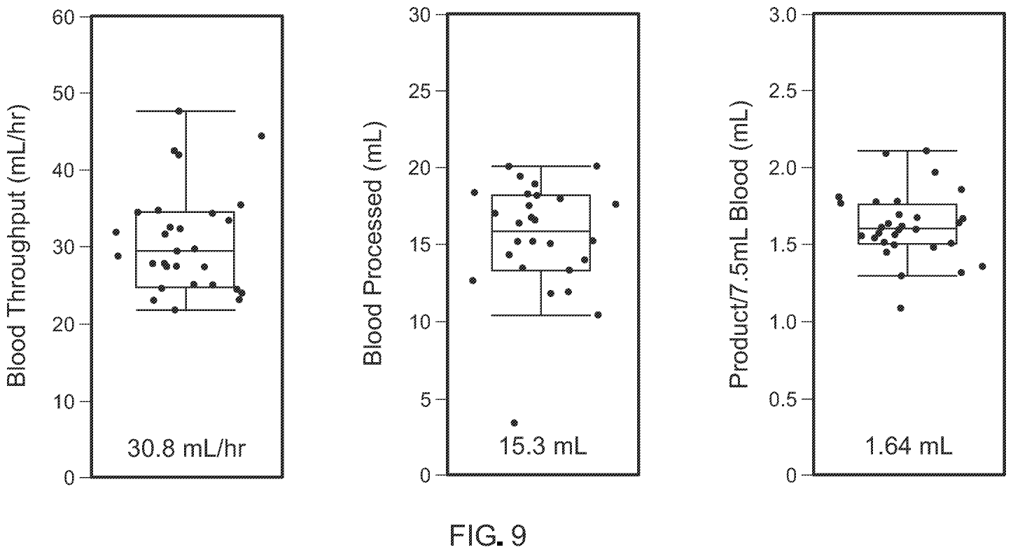

During operation, sample fluid and buffer are pushed through the microfluidic device in various two ways. In some instances, syringe pumps with specified input flow rates are used to introduce sample fluid and buffer into the microfluidic device. In other instances, a single pressure source (e.g., a 60 mL plastic syringe) is connected to sample and buffer inlet reservoirs. Use of pressure control enables rocking of a blood sample to avoid settling during the run. Flow rates internal to the microfluidic device are controlled passively by built-in fluidic resistors. Blood sample viscosity is variable even with the normalization of hematocrit (HCT) to 20. As depicted in volumetric splits exhibited good reliability during pressure-driven experiments when compared to the design split targets (depicted in FIG. 8). These included the flow fraction entering the product of each NISA stage as well as the injection fraction of sample to stage 130. Processed blood volume averaged 15.3 mL, blood flow rate was 30.8 mL/hour, and product volume was 1.64 mL per nominal tube of blood (7.5 mL). Data reported for 30 runs are depicted in FIG. 9.

### C. Techniques for Isolating Rare Cells Using Permeability-Enhanced Ultra-High-Throughput Magnetic Sorting Devices

a. Design

Computational finite element modelling of magnetic fields can be applied to select the appropriate width of high permeability channel channels 214 and 226. This technique permits maximization of the magnetic field gradient applied to sorting channels 212 and 224 (depicted in FIG. 2A). Microfluidic devices with sorting channel dimensions (channel widths varied between 1000-2000 μm, and sorting channel heights varied between 50-100 μm) were examined to identify an optimal design for the microfluidic device 200 (e.g., a sorting channel with the largest magnetic field gradient applied). In some implementations, the sorting channel 212 has a channel width of approximately 1500 μm, and the sorting channel 224 has a channel width of approximately 1800 μm and a channel height of approximately 60 μm. In such implementations, the highest depletion of WBCs is achieved without losing more than 2% of spiked CTC lines.

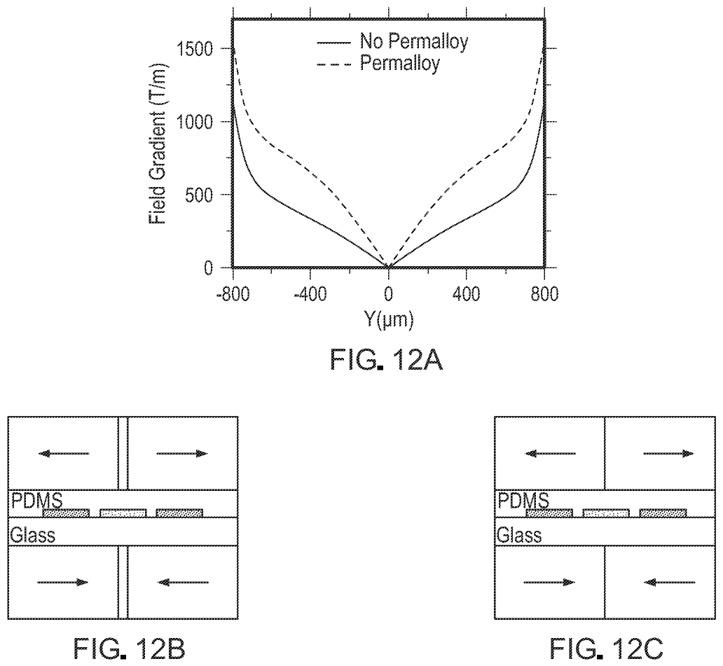

In some implementations, magnetic permeability strips of material with a relative magnetic permeability of approximately 10 or more (e.g., nickel, iron, ferrites) can be placed underneath the sorting channel and between adjoining permeability channels to intensify the deflecting magnetic field generated by magnetic permeability particles within the permeability channels (depicted in FIG. 12A). For example, the magnetic permeability strips can be permalloy strips made from a nickel-iron magnetic alloy. In some instances, the magnetic permeability strips can be configured to increase the magnetic field gradient in the bulk of the sorting channel by up to approximately 50% (relative to the magnetic field generated without the use of magnetic permeability strips). Magnetic field modeling can be performed using COMSOL to study the effect of high gradient channels. For example, FIG. 12A shows that the presence of high permeability soft iron in the vicinity of the sorting channel leads to 40-fold higher magnetic gradient as compared to the other magnetic circuits.

In operation, a sample fluid including magnetically labeled cells and a buffer solution can be introduced into the microfluidic device 200 using syringe pumps or a pressure source (e.g., a 60 mL plastic syringe) connected to the sample or buffet inlet reservoirs. The syringe pump can be configured to provide a specified input flow rate. Use of pressure control allows rocking of magnetically labeled cells, which prevents settling of cells in sorting channels 212 and 224. Flow rates internally within sorting channels 212 and 224 can be controlled passively by built-in fluidic resistors.

b. CTC Separation from Leukapheresis Samples

FIG. 2B is a schematic showing a microfluidic approach for isolation of untouched CTCs from leukapheresis products. A typical 65 mL leukapheresis product derived from differential centrifugation of approximately 5 L of whole blood consists of 3 to 6 billion WBCs, with 10 to 30 billion contaminating RBCs. Leukopaks® from patients with cancer are can also have 100 to 20,000 CTCs, depending on the type and stage of malignancy. WBCs consist primarily of mono-nuclear cells, since neutrophils are depleted by centrifugal forces during apheresis, and their concentration within the Leukopak® ranges from 50 to 90 million cells/mL, >10-fold higher than the WBC concentration in whole blood. Depending on the apheresis settings, the concentration of contaminating platelets may also be 10-fold higher in a Leukopak®, compared with whole blood. Altogether, the very high number of WBCs and platelets, concentrated within a large volume of leukapheresis product, present challenges, compared to standard 10 mL samples of peripheral blood that are currently used for microfluidic enrichment of CTCs.

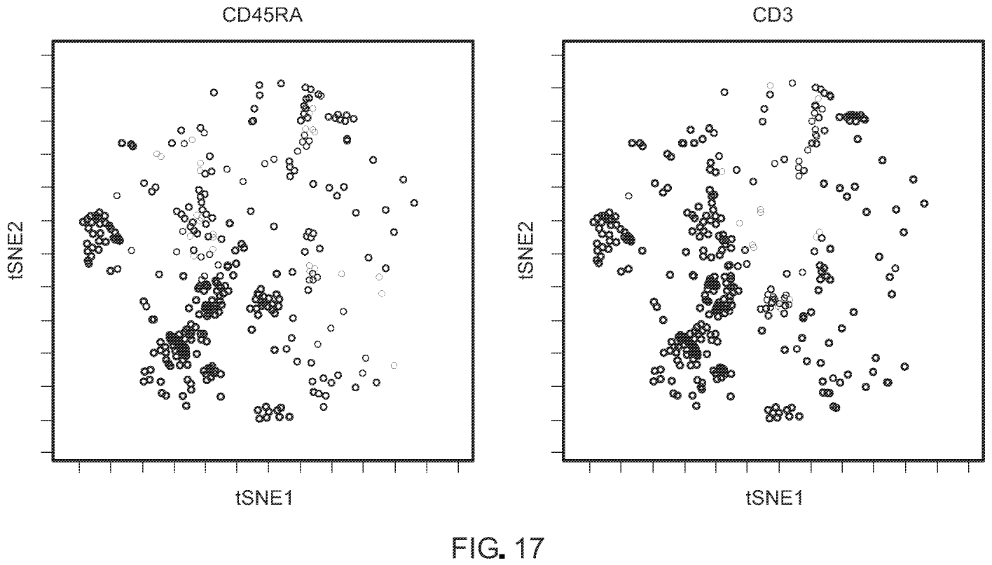

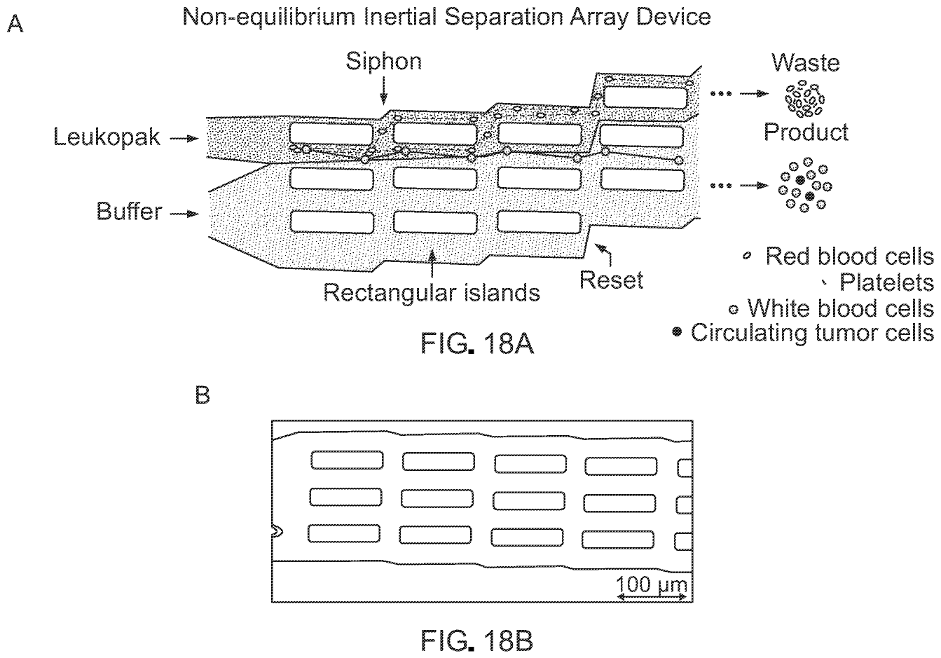

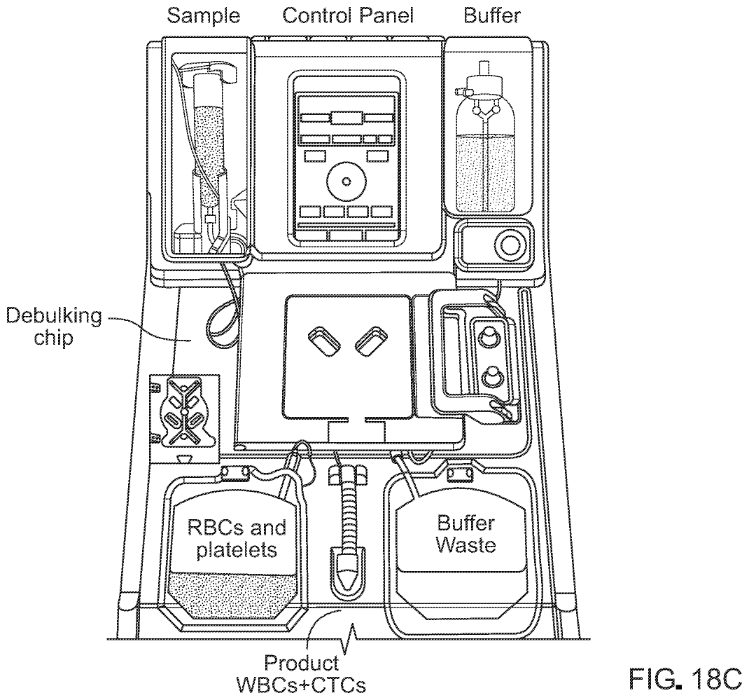

The workflow depicted in FIG. 2B can be used for depletion of hematopoietic cells from leukapheresis products. WBCs are initially labeled with a cocktail of biotinylated antibodies targeting the pan-leukocyte cell surface antigens CD45, CD16, CD3, CD45RA, and CD66b. A non-equilibrium inertial separation array can be used to remove RBCs and platelets based on their small physical size, compared with nucleated cells. CD3 and CD45RA antibodies can be added to further deplete WBCs, based on mass cytometric profiling of contaminating cells in the product (as depicted in FIG. 17). A non-equilibrium inertial separation array can be used to remove RBCs and platelets based on their small physical size, compared with nucleated cells (as depicted in FIGS. 18A and 18B). Antibody-bound WBCs are tagged with 1 μm streptavidin-coated superparamagnetic beads and a high-throughput magnetic sorter chip is then used to deplete WBCs and recover unlabeled CTCs. In some instances, the CTC isolation process using the workflow depicted in FIG. 2B can be completed within three hours.

### EXAMPLES

### A. Experimental Evaluation of Size-based Sorting Microfluidic Devices

a. Cell Size Cutoff

To verify the size cutoff of the size-based sorting microfluidic device (depicted in FIGS. 1A and 1B), model experiments were run using two CTC lines (MGH-Mel-182-1 and MGH-Pem-22). These cell lines were isolated from blood samples of two patients bearing metastatic melanoma that were treated at Massachusetts General Hospital. The cells were chosen because their size distributions were centered around the intended size-cutoff for sorting (depicted in FIG. 3C for MGH-Mel-182-1). MGH-Mel-182-1 suspensions had a median diameter of 16.3 μm, and MGH-Pem-22 suspensions had a median diameter of 18.8 μm (Z2 Coulter Counter Analyzer, Beckman Coulter). Instead of blood, 7% Ficoll PM 70 was used as the sample matrix. This simulated the viscosity of 1:1 diluted whole blood without obscuring spiked CTCs in the waste, enabling yields to be measured.

The results are experiments are depicted in FIG. 3B. Cell size for 90% yield is plotted against per-array flow rate (syringe pump control). To get each data point, cell diameter distributions were first measured in product (FIG. 3D) and pooled waste (FIG. 3E), again by Coulter counter. Next, yield of cells was quantified as a function of diameter within 1 μm wide bins (FIG. 3F), and the cell diameter with 90% yield was estimated by curve fit. The overall fit in FIG. 3B is linear with flow rate (semi-log x-axis results in apparent non-linear trend) and exhibits the expected drop-off with flow rate (inertial focusing increases at higher Reynolds number).

To minimize shear stress while keeping high cluster yield, a per-array flow rate of 200 μL/min was used for remaining experiments, requiring a 10-psi input pressure to sample and buffer. With sample viscosity of 2 mPa-s, this flow rate resulted in a particle Reynolds number of about 0.1 for cells of diameter (a) 18.5 μm (the 90% cutoff); therefore, inertial focusing 34 is actively pushing cells near the size cutoff across streamlines. In this calculation, particle Reynolds number (Rep) is defined as Re*(a/Dh)² where Re is the channel Reynolds number (calculated as pVDh/μ). Additionally, ρ and μ are mass density and dynamic viscosity of the fluid, respectively, and V is mean flow velocity, and Dh is the hydraulic diameter of the focusing channel depicted in the left side of FIG. 3A.

b. High Flow Rate Analysis

Although size cutoff could be reduced further at higher flow rates, the use of PDMS as a device material causes channels to inflate from the increased pressure. This increases channel width and requires cells to migrate even farther from the wall to remain in the product lane of a given NISA module. Therefore, to examine NISA performance at much higher flow rates, whole blood was injected into a single isolated NISA module (100% injection).

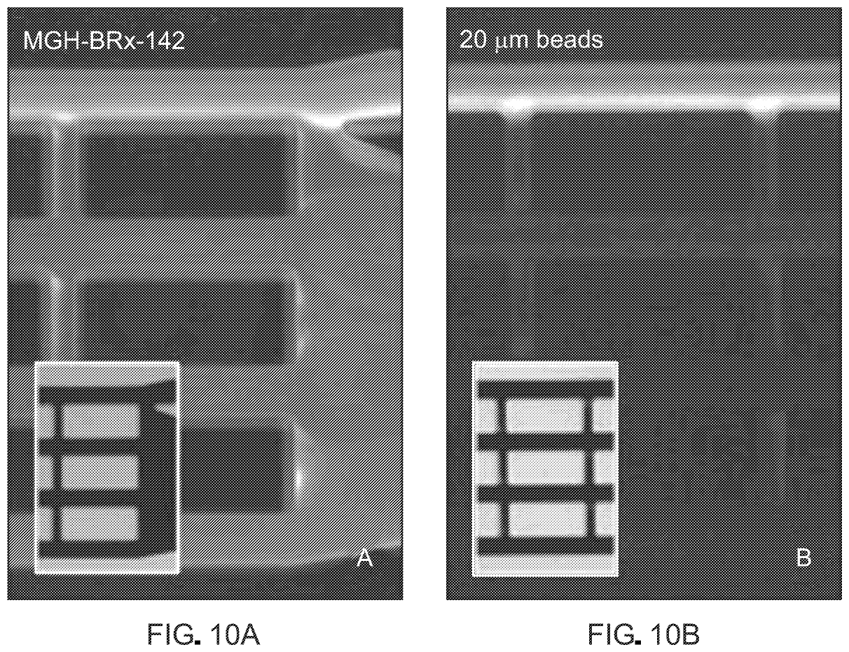

The results of this experiment are depicted in FIGS. 10A and 10B. The results confirm that CTCC sorting (MGH-BRx-142 cells) is possible even at 2 mL/min per array of whole undiluted blood (47% HCT), and that a single array can process 20 mL of whole blood (35% HCT) four times at 4 mL/min. Based on this data, a size-based sorting microfluidic device (e.g., microfluidic devices 100, 150) could process as much as 320 mL of whole blood in 20 minutes. This extreme throughput is particularly notable given that the NISA modules have no filter to remove debris. Rather, they act as a continuous flow size-sorter that almost never clogs. A multi-stage microfluidic device operating this fast may require a rigid material like plastic to avoid inflation, and shear stress would be too high for intact cluster isolation. An alternative approach could be to run blood slowly at first (e.g., at 200 μL/min per array) to remove larger CTCs and clusters gently, then increase the flow rate to as high as 2 mL/min to isolate smaller single CTCs from the waste fractions.

c. Blood Cell Removal

Removal of blood cells (and smaller CTCs) (i.e., depletion) is also important to the isolation of larger CTCs and CTCCs. To measure depletion performance, a set of experiments were performed using a blood intended sample matrix. Specifically, 17 chips were run, processing 15 mL whole blood each through the microfluidic device 150 depicted in FIG. 1C. To standardize the viscosity and more precisely measure depletion at a controlled flow rate, blood was diluted to 20% hematocrit by a buffer (1% w/v F127 in PBS). This is about 1:1 dilution on average. After processing through the chip, platelets (PLTs), RBCs, and WBCs were counted manually in the product using hemacytometers and Nageotte chambers depending on the cell concentrations. Vybrant DyeCycle green was used to label WBCs, while Calcein AM allowed platelets to be identified.

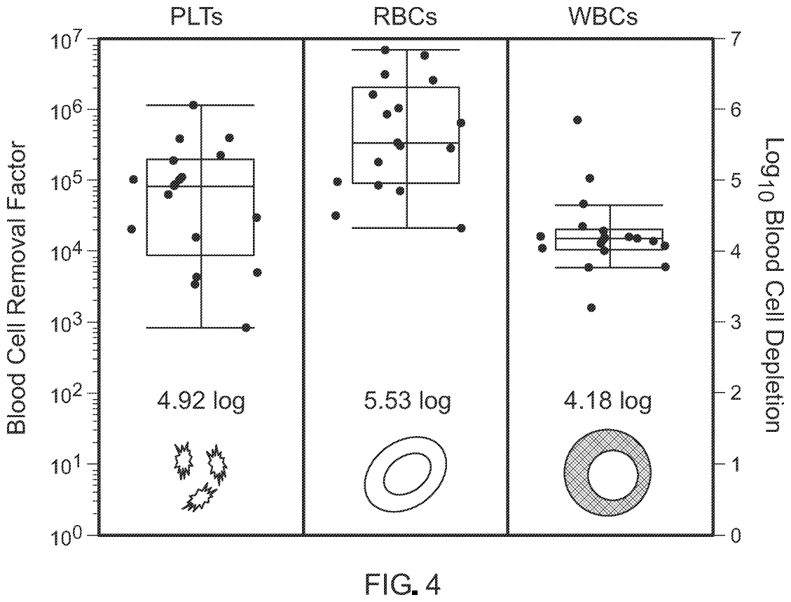

Blood cell removal factor is defined for each cell type as total cells injected to the chip divided by total cells found in product. This is depicted in FIG. 4 alongside the base-10 log depletion equivalent (given as a summary statistic). Briefly, platelets were removed to a level of 12 ppm (4.92 log 10), red blood cells to 3 ppm (5.53 log 10), and WBCs to 66 ppm (4.18 log 10), where 1000 ppm indicates 99.9% removal (3-log depletion).

Though this is also a higher level of cell depletion in each case than reported for other microfluidic chips where depletion of platelets, RBCs, and WBCs is 4, 5, and 4 respectively (based on internal data), it still leaves about 3600 platelets, 15 thousand RBCs, and 400 WBCs in the product per mL of blood processed (based on a standard blood sample with 300 million platelets, 5 billion RBCs, and 6 million WBCs per mL). It would be possible to improve these depletion levels by re-running the product through additional NISA modules. Platelet removal is complicated by their association with WBCs (satellites) and self-aggregation in some donors, so platelet removal may remain below that of RBCs despite their smaller size individually.

d. Gentle Isolation of CTCCs

Techniques for CTCC isolation typically should not break up cell clusters that exist in the bloodstream. To accomplish this, CTCCs should not be subjected to fluid stresses in excess of what may occur in the body, though transit through a microfluidic device is often shorter than residence in circulation. Flow acceleration is particularly suited to break cell clusters (data not shown), so the design depicted in FIG. 1A avoids any rapid constrictions while also keeping shear stresses below 15 Pa.

To determine if CTCC isolation using the size-based sorting microfluidic device was gentle to potentially fragile CTCCs, spiked CTCCs were observed at all key decision points within the three stages (depicted in FIG. 1A). Specifically, the following points were observed: inlet points 152 (just after the sample inlet port), outlet of NISA modules 152A-D (stage 1), outlet of NISA module 154A (stage 2), and the outlet of NISA module 156A (stage 3). To ensure that CTCCs were clearly visible within the sample and waste streams, Ficoll PM 70 (7% w/v in 1% F127 in PBS) was used as a blood sample viscosity mimic. Besides keeping shear stresses comparable to that for 20% HCT blood, this also ensured volumetric splits remained within desired ranges. High speed videos at each location were manually examined to count individual spiked CTCs and CTCCs of varying cell number. This technique enabled a verification that CTCCs conserved their integrity throughout the microfluidic device.

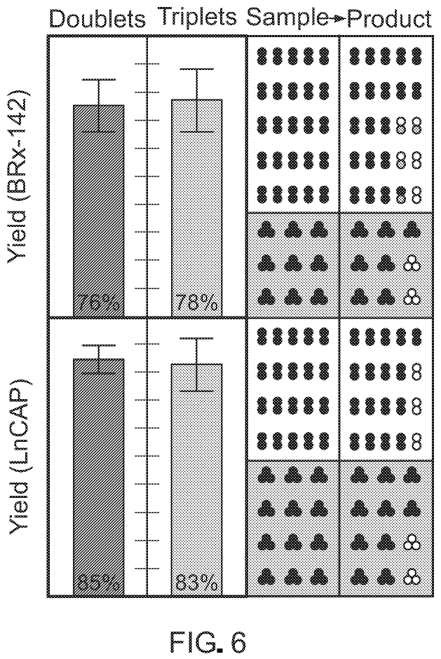

The experiment was performed for two separate cell types: (i) MDA-MB-231 (ATCC), a standard triple negative breast cancer cell line, and (ii) MGH-BRx-142, a cultured line of CTCs directly derived from the blood of a patient with hormone receptor positive breast cancer. The cell type MGH-BRx-142 cells was chosen in part since clusters are fragile (as will be apparent from FIG. 6), often breaking up during insufficiently careful pipetting. The cell type MDA-MB-231 cells were chosen in part because they are a commonly used cancer cell line for spike cell experiments, allowing outside comparison. In each case, cultured cells were injected to the microfluidic device unaltered, and no special handling was performed to artificially cluster the cells. For MGH-BRx-142 input suspensions, single cells represented 65% of the total with the remaining cells residing in clusters of 2.8 cells each on average. For MDA-MB-231 input suspensions, single cells represented 64% of the total with the remaining cells residing in clusters of 2.9 cells each on average.

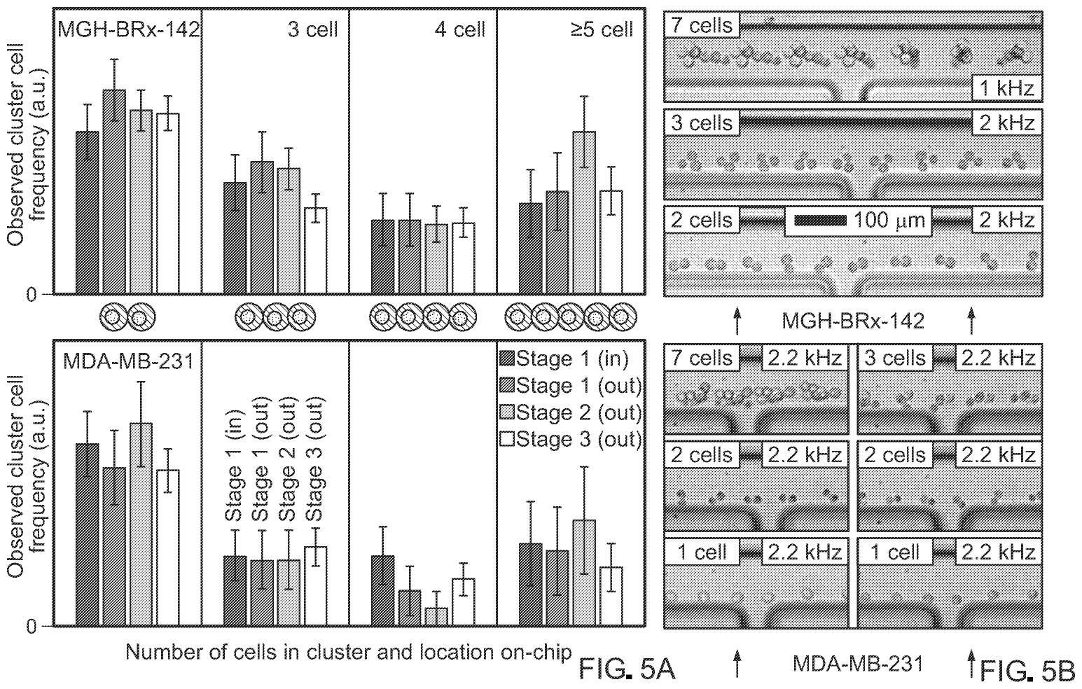

Observed cluster distributions are depicted in FIG. 5A. Large CTCCs of 5 or more cells are lumped together to improve sampling error in less frequent event classes. Cluster cells were tallied per cell, so that one five-cell cluster plus one 10-cell cluster counted as three give-cell clusters when plotted. No CTCCs were observed to enter the waste path in any of the three stages for either spike cell line, or output cluster size distributions closely matched the inputs. Specifically, 146 MGH-BRx-142 clusters were observed entering the product output (2.7 cells/cluster on average), and 427 MDA-MB-231 clusters were observed entering the particle separation module 130 product (2.9 cells/cluster on average). Examples of CTCCs that transitioned into the product (in the third stage) are shown on the right side of FIG. 6, which highlights rotation and orientation of CTCCs.

FIG. 5B shows that magnitudes of each population relative to the others (within a cell line) are proportional to input spike number (though the actual magnitude is arbitrary). MGH-BRx-142 cells 35 are relatively large (median diameter of 19.3 μm), and their yield of single CTCs from input to stage 3 product was 97.6%. MDA-MB-231 cells exhibited a yield of 76.4% to product at 200 μL/min per array. Moreover, successful isolation of one hundred seventeen MDA-MB-231 doublets was observed in a row at the stage 3 product, albeit in a blood viscosity mimic to enable observation.

e. Rare CTCC Isolation from Blood

The end-to-end yield of small numbers of manually spiked CTCCs using size-based sorting microfluidic devices (e.g., microfluidic devices 100, 150) was also tested using whole blood. Specifically, either five 2-cell CTCCs or three 3-cell CTCCs were spiked into 15 mL of whole blood, mixed with buffer (1% w/v F127 in PBS) to 20% hematocrit, and processed through the chip using pressure control (10 psi). In comparison with all other spike cell experiments, where cell suspensions were mixed into Ficoll or blood samples, direct cluster spiking was performed here by micro manipulating individual fluorescent pre-labeled clusters and transferring them into whole blood. After isolation in the microfluidic device, CTCCs were identified in the whole product volume by panning a microscope objective throughout a well plate reservoir.

The results of 16 cluster-spike experiments are depicted in FIG. 6 across two cell lines. Though cluster yield within the microfluidic device is expected to be very high, experiments incurred variable transfer losses in manually ejecting spike clusters from the glass cell-picking capillary. This is because a gentle flow was required to avoid breaking clusters even at the point of spiking into blood, yet that risked being insufficient to dislodge partially adhered clusters. Nevertheless, around 77% of MGH-BRx-142 clusters and 84% of LnCAP clusters were found intact in the product, demonstrating the ability to find a handful of CTCCs in an admix of about 75 billion RBCs, 90 million WBCs, and 4.5 billion platelets. In these experiments, MGH-BRx-142 cells were again chosen in part due to their observed fragility as CTCCs, and LnCAP cells (ATCC; prostate cancer cell line) were chosen as a common cell line to serve as a point of comparison.

f. Spiked Cell Growth Post-Isolation

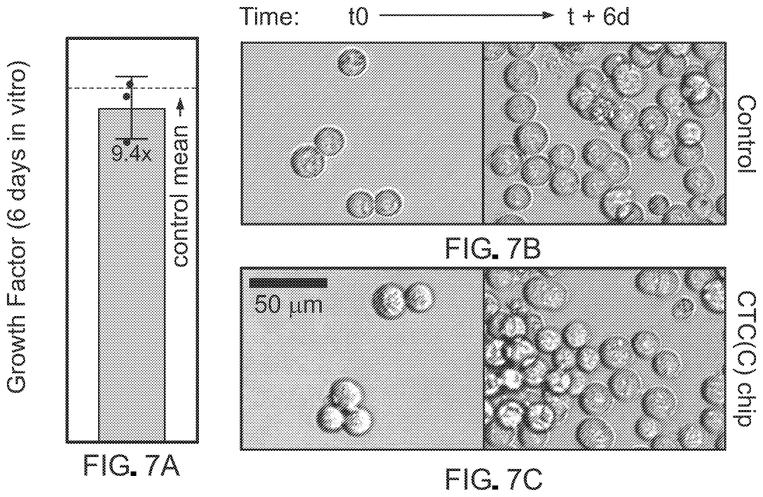

Besides isolation of intact CTCCs, further testing also performed to determine whether isolation would affect growth rate of isolated spike CTCs and clusters. MGH-BRx-142 cells were spiked using a CTC line derived from breast cancer patient blood into three 15 mL blood aliquots. The product fractions were cultured for six days. When compared to day-matched controls of equal input spike number, isolated spike CTCs and clusters grew by about 9.4-fold for six days in vitro, which was found to not be significantly different compared to for matched controls (depicted in FIGS. 7B and 7C). This cell line was chosen in part because it is relatively large (19.3 μm median by Coulter counter), nearly removing isolation yield from the comparison, leaving just growth rate as a driver of final cell numbers.

g. CTCC Types

Experiments were also conducted to evaluate how size-based sorting microfluidic devices (e.g., microfluidic devices 100, 150) are positioned to capture each subtype of CTCCs. The characteristics that often affect size-sorting are the number of cells in the cluster (e.g., two to 50 is typical), overall cell size (most are 20 to 130 μm, but some may be greater than 300 μm), and the strength of intercellular adhesions. CTC doublets composed of small individual cells are closest in size to WBCs (though doublets including one CTC and one WBC might be even smaller). In buffer (or Ficoll), cell doublets were found to almost always tumble end over end as depicted in FIGS. 5A and 5B. This side-effect of inertial focusing helps increase their effective size by pushing their center of mass further from the channel wall than if they were to spin along their long axis. FIG. 6 demonstrates the ability to isolate spiked CTC doublets. As CTCC size increases, size sorting is easier but actual forces applied to the cluster gradually increase as the CTCC occludes more of the channel. Therefore, it was important to increase channel width to 100 μm and reduce shear stress at the wall to near the physiological level.

Results of isolation of the largest CTCCs (referred to as “CTMs”) can also be extrapolated. Recently, inertial migration was applied in a straight microchannel to separate CTCCs from buffy coat or blood. As described herein, a sample is injected at about 50 μL/min (1:2 to buffer) from each side of a 150 μm wide, 50 μm high, microchannel. After 2 cm, cells over 14 μm migrate into the central buffer flow, leaving most blood cells behind. Despite that sample passes through a twice-narrower sorting channel, CTMs of apparent size up to 340 μm were found in samples of head and neck cancer patient blood. This suggests the microfluidic device 100 can capture even larger clusters, and by reducing wall shear stress over ten-fold, weaker bound clusters. These large CTCs contained WBCs, as observed bound to CTCCs in other studies. In addition, CTCs were sometimes observed with platelet satellites, and platelets have been linked to protection of CTCs against immune targeting by natural killer cells. Size-sorting does not distinguish among these differences, so each cluster type should be found by size-based sorting microfluidic devices.

h. CTC and CTCC Isolation in Clinical Samples

Cultured CTCs (used in spiking experiments) differ from CTCs and CTCCs present in patient blood samples (i.e., clinical samples). Patient CTCs can be smaller than many cultured CTCs and are also often apoptotic. Given such differences, the fraction of primary CTCs and CTCCs isolated by size-based sorting microfluidic devices (e.g., microfluidic devices 100, 150) should be evaluated using clinical samples.

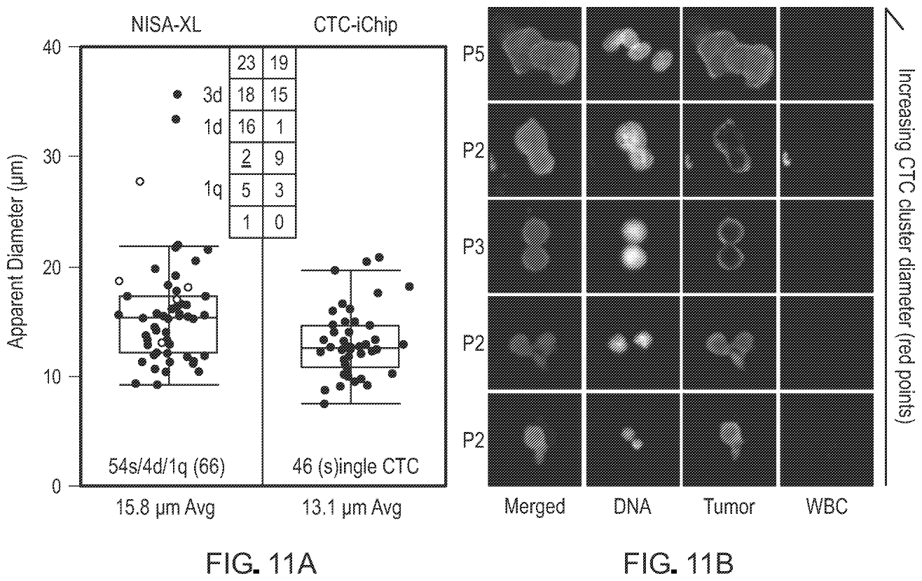

In one experiment, blood samples from six melanoma patients (18 mL blood per patient) were processed using two size-based sorting microfluidic devices. FIGS. 11A and 11B show results of isolation of this experiment. The first microfluidic device is referred to as “NISA-XL” and includes features similar to those discussed in reference to microfluidic devices 100 and 150. The second device is referred to as “CTC iChip” and includes features similar to those previously disclosed in U.S. Pat. Nos. 9,610,582, 9,895,694, and 10,150,116.

To isolate smaller CTCs that were missed by size sorting alone, device waste fractions were processed using negative selection (CD45, CD16, and CD66b targeted magnetic beads). Immune fluorescence (IF) imaging at 10× was then used to find putative CTCCs.

A total of 66 CTCs were found by the microfluidic device (including 4 CTC doublets and one four-cell CTCC). A total of 46 were found by the CTC-iChip, all of which were single CTCs. In total, this amounts to 1.0 CTCs per mL blood, similar to our prior results in melanoma 1. Notably, 11% of all putative CTCs were in CTCCs, and CTCCs were found in 3/6 patients. In all, 59% of CTCs were found by the NISA-XL chip (54% of single CTCs and all CTCCs), with the remaining single CTCs being found in the CTC-iChip product. The dataset includes an experiment where bubbles were observed in the stage 3 NISA-XL device (18% yield of single CTCs in patient 4). These fractions will change with patient cohort, and some CTCs or CTCCs in the NISA-XL waste may be missed by the CTC-iChip. Yet because many patient-derived CTCs overlap in size with WBCs 1, one would expect to lose a fraction by size sorting alone, consistent with these results.

i. Melanoma CTC Immunofluorescence CTCs and CTCCs were isolated from clinical samples using two size-based sorting microfluidic devices (results depicted in FIG. 11A). Products collected from size-based sorting microfluidic devices were fixed (0.5% PFA, 10 min), plated on slides using the Shandon EZ megafunnel (Thermo Fisher) and Cytospin cytocentrifuge (2000 rpm, 5 min), then permeabilized (0.3% Triton X-100, 45 min). Tyramide signal amplification (TSA staining system, Akoya Biosciences) was used to amplify multi-color IF, where unconjugated primary antibody was bound to target antigens, and secondary antibodies conjugated to horseradish peroxidase were then added to bind the primaries (incubation with TSA reagent adds the fluorophore). In this manner, leukocytes and CTC(C)s were labeled with a red exclusion channel (AlexaFluor 594) and green inclusion channel (AlexaFluor 488) respectively.

Specifically, CD45 was used to identify WBCs (primary: 3H1363 clone, Santa Cruz; secondary: polyclonal, Jackson ImmunoResearch). Three melanoma cell targets were pooled in green to identify CTCs: premelanosome protein (PMEL), tyrosinase, and chondroitin sulfate proteoglycan 4 (CSPG4 or NG2). PMEL is one of the structural components in the premelanosome (primary: HMB45 clone, Dako; secondary: polyclonal, Jackson ImmunoResearch). Tyrosinase is an enzyme regulating tyrosine metabolism in melanin synthesis (primary: T311 clone, Dako; secondary: polyclonal, Jackson ImmunoResearch). CSPG4 has been associated with melanoma 2 (primary: LHM-2 clone, R&D Systems; secondary: polyclonal, Jackson ImmunoResearch). Finally, DNA was labeled with DAPI. Putative CTCs were identified at 10× magnification as inclusion-positive, exclusion-negative, nucleated events, and clusters were rescanned at 60× using multi-spectral imaging (Vectra, PerkinElmer) to obtain images at right in FIG. 11B. The measured diameter of each CTC and cluster was computed from a drawn mask of the merged 10× image (ImageJ) by finding the diameter of a circle that has the same total area. It is important to note that after plating and staining, putative CTCC geometry can be different than diameter under flow. A difference was observed in average size (shown on lower left of FIG. 11B).

### B. Experimental Evaluation of Permeability-Enhanced Ultra-High-Throughput Magnetic Sorting Devices

An example of a permeability-enhanced ultra-high-throughput magnetic sorting device is depicted in FIG. 2A. Experiments were conducted to evaluate the use of immunomagnetic sorting on CTC and CTCC isolation from blood samples. Results of the experiments showed that the device operated at a blood equivalent throughput of 100 mL/h and was capable of removing WBCs labeled with 0.5 μm beads.

In the first stage of sorting (shown on the left side of FIG. 2C), two inertial focusing channels 208 feed labeled cells in a sorting channel 212 via asymmetric serpentine channels. At the core of the channel, a buffer flow is provided from buffer inlet 210 to keep the cell stream close to the channel wall, where magnetic field gradients are at maximum. For example, as depicted in FIG. 12A, field enhancement due to magnetic permeability strips, such as permalloy strips, between magnets. In most of the sorting channel, field gradient increases due to inclusion of magnetic permeability strips between magnets.

As WBCs move through the sorting channels, cells with greater than 30 particles are deflected to the center of the channel and exit through the waste outlet port 216. This provides a clog-free design where WBCs are deflected into the core of the flow away from the walls. Greater than 99% WBCs were identified to be removed in the first stage itself. Almost all the free unbound beads were also removed in the first stage. Remaining cells with fewer attached beads and CTCs enter the second stage, where a pair of inertial concentrator channels 220 achieved 15x concentration, and placed cells very close to the channel wall. High magnetic gradients in the second stage pushed tagged WBCs into the center of sorting channel 224 to remove more than 99.9% WBCs.

Overall, at the product outlet 230, the microfluidic device 200 achieved 25x concentration and CTCs from 200 mL blood were collected in a buffer volume of 8 mL. The microfluidic device 200 was also tested by spiking cancer CTCs in buffy coat generated from healthy donor blood samples. On average, results showed that the device removed 99.95% of WBCs and recovered more than 95% spiked CTCs.

a. Depletion of WBCs

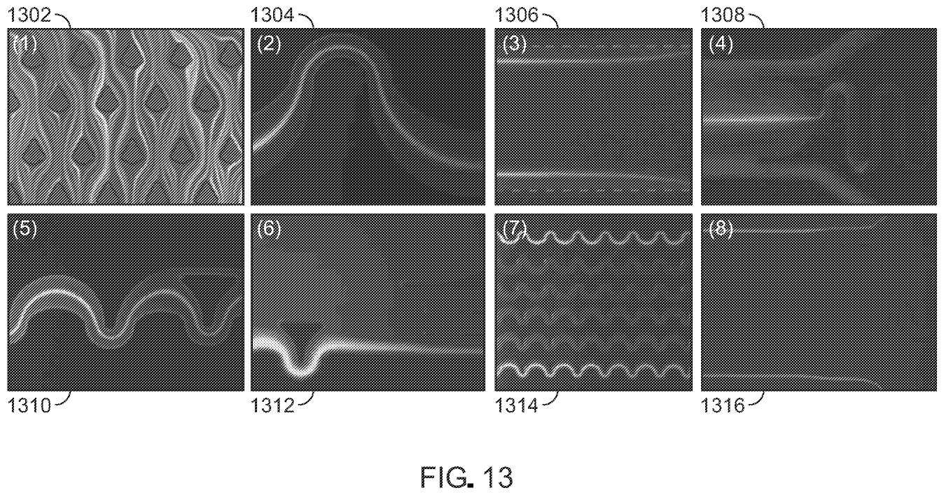

FIG. 13 shows fluorescence streak images of WBCs at various positions of the microfluidic device depicted in FIG. 2A. A magnetically labeled cell suspension initially flows into the first stage of sorting through two sets of microfluidic inlet filters (e.g., filters 204, 206), which, for example, can have 40 μm apertures to remove large debris or aggregates, as depicted in 1302. Slender debris with diameter smaller than the aperture of filter-1 (e.g., filter 204) are captured based on their length in the tortuous channel geometry of filter-2 (e.g., filter 206). After the filters, the cell suspension flows into stage-1 sorting channel (e.g., channel 212) at 48 mL/h via two asymmetric serpentine channels (e.g., channels 208), which inertially focus cells in a single file, as depicted in 1304. These serpentine channels utilize a balance between shear-induced lift force and Dean flow-based drag force to focus cells near the center of the serpentine channel. At the core of the sorting channel, a buffer flow at 120 mL/h is provided to keep the inertially focused cells close to the channel wall, where magnetic field gradients are at maximum, as depicted in 1306. As WBCs move through the deflection channel, they experience a magnetic force and are deflected toward the center of the channel into the stage-1 waste port, as depicted in 1308. This provides a clog-free design, where WBCs are deflected into the core of the flow away from the walls and high gradient regions.

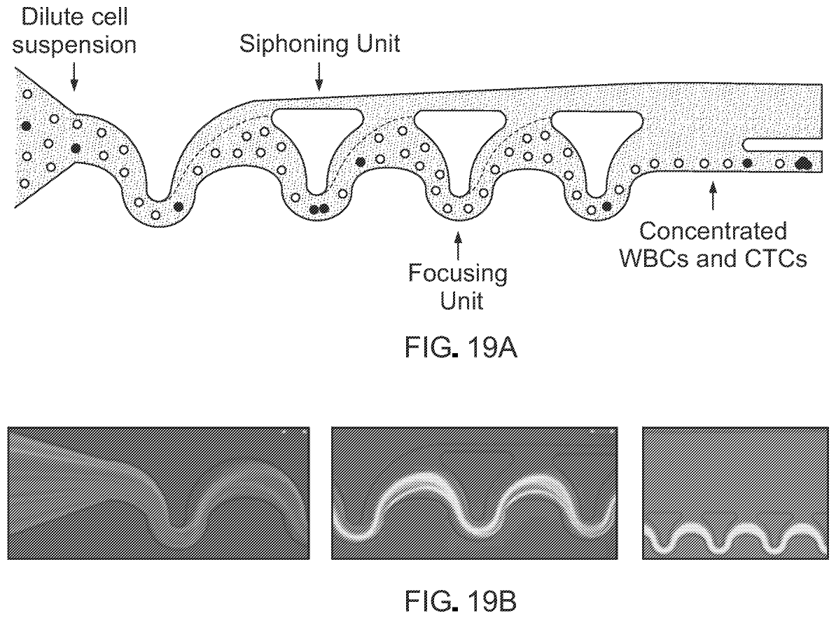

After the first stage of sorting, the cell suspension flows into the second stage of sorting via another microfluidic filter (e.g., filter 218) with, for example, a 40 μm aperture and two inertial-focusing based cell concentrators. The cell concentrator works by continuously creating a cell-free region and repeated siphoning using passive flow-controlled resistance, as depicted in 1310. Cells pass through asymmetric-inertial focusing units, which create a cell-free region due to inertial lift forces and Dean-flow-induced drag force (depicted in FIG. 19A). This cell-free region is siphoned away from curved focusing units by a siphoning channel, while cells pass through another focusing unit that creates a new cell-free region, which is siphoned again. This process can be repeated over 140 units until the end of the channel achieving ˜20-fold concentration of cells, as depicted in 1312.

The siphoning unit serves two key purposes. First, by concentrating the cells, they are positioned close to the walls in the stage-2 sorting channel (e.g., channel 224) where magnetic gradients are maximal. Second, after concentration, the excess cell-free fluid is removed through a stage-2 waste port (e.g., waste outlet port 216). This reduces the net flow input into the stage-2 sorting channel and provides a greater residence time for cells. Six-feeder channels supply the concentrated cell-suspension to the stage-2 sorting channel where any loss in cell focusing is corrected by six inertial focusing units and cells are placed in a single file close to the channel sidewall, as depicted in 1314.

In the stage-2 sorting channel, every cell labeled with a bead is deflected to the waste port, while undeflected CTCs are collected within an 11-fold reduced volume, as depicted in 1316. FIG. 2D shows an image of the microfabricated polydimethylsiloxane (PDMS) magnetic sorter microchip with filled high-permeability channels. In some instances, the microchip can achieve a total flow rate (buffer and sample) of 168 mL/h.

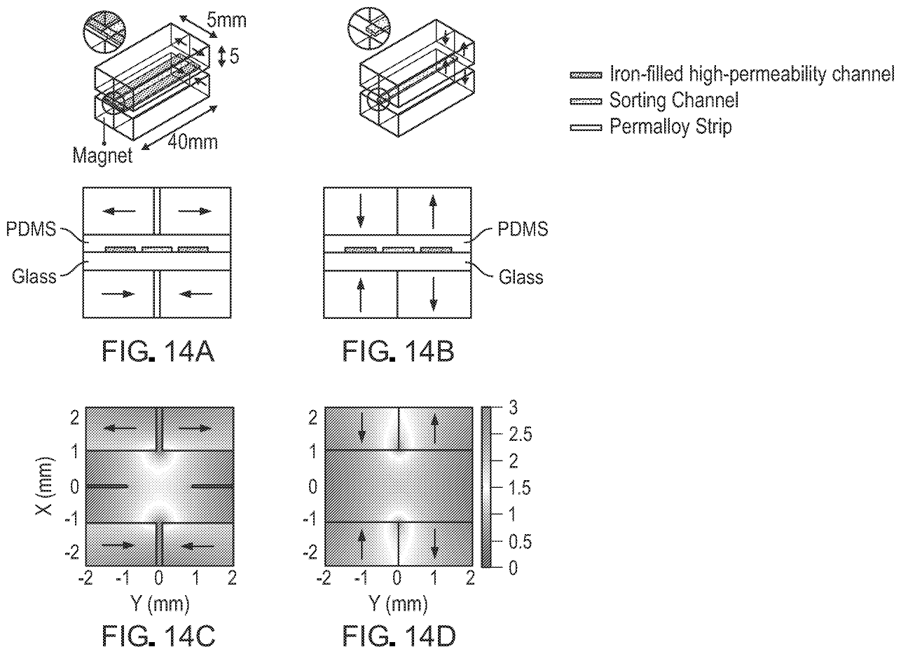

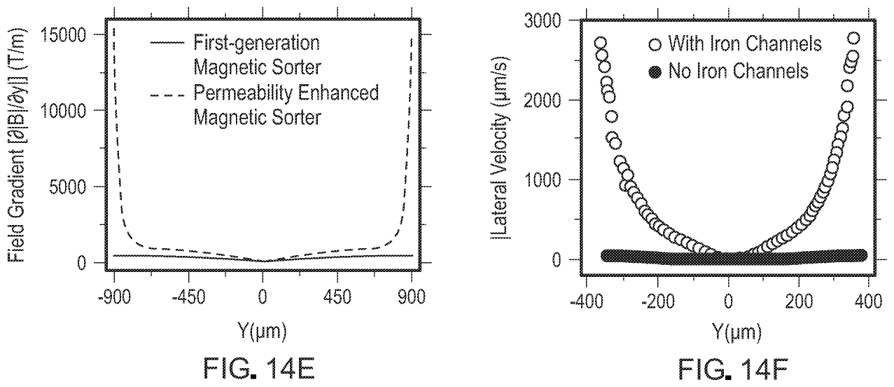

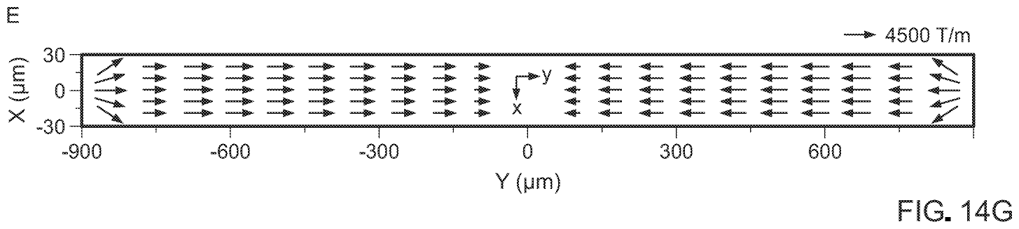

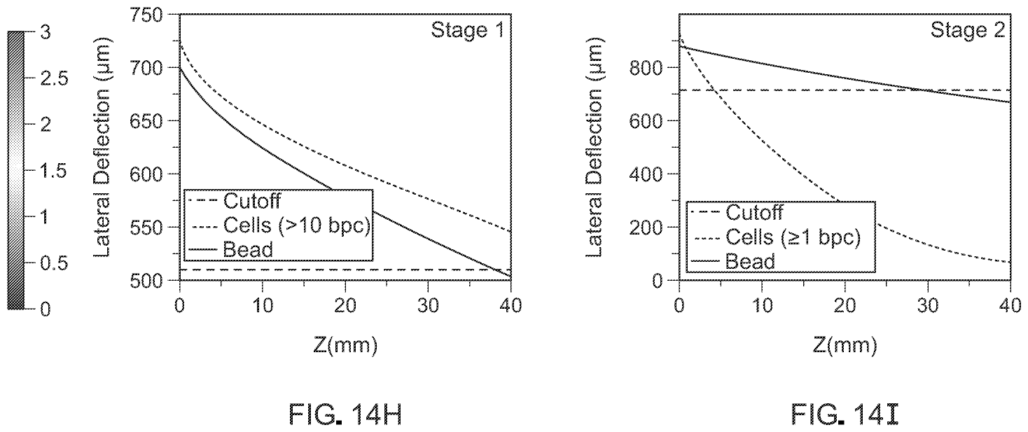

b. Permeability-Enhanced Magnetophoresis The ultra-high throughput functionality of the magnetic sorter is achieved using a permeability-enhanced magnetic configuration depicted in FIG. 14A. The configuration includes a quadrupolar arrangement of rectangular (e.g., 5 mm×5 mm×40 mm) neodymium-iron-boron (e.g., N52 grade) magnets. The polarity of magnets can be modified to the y-direction to ensure that the magnetic force on the cells is directed toward the center of the sorting channel in the presence of the adjoining iron-filled channels.

If superparamagnetic particles used to label cells are saturated, the lateral magnetic force on a labeled cell is directly proportional to the number of particles attached to a cell, and, the gradient of the norm of the magnetic field in the y-direction. The magnetic field gradient can be increased to increase throughput and thereby achieve deflection of a cell labeled with a single bead. As depicted in FIG. 14A, this is accomplished using high-permeability channels that are filled with soft magnetic iron particles with a 100 μm thick permalloy strip between magnets. Under the action of the macro magnetic field from the rectangular magnets, the channels become magnetized and produce a localized magnetic field that decays rapidly and results in a high magnetic field gradient in the sorting channel. A long-range field gradient from rectangular magnets is also present in the sorting channel though it is smaller (e.g., 35-fold smaller). The channels can then act as on-chip magnetic micro-lenses and significantly increase the magnetic field gradient. As depicted in FIG. 14E, this configuration creates a field gradient as high as 15,400 Tesla/m in deflection channels as compared to the 440 Tesla/m previously achieved by a first-generation magnetic sorter. In this example, the configuration depicted in FIG. 14A produces a 35-fold enhancement in magnetic force.

FIG. 14F is a graph that includes experimental results of measuring lateral deflection velocity of 2.8 μm superparamagnetic tracer particles with a high-speed camera. The lateral velocity of magnetic particles is directly proportional to the field gradient. A 4-mm viewing gap was created between magnets for direct high-speed imaging of particle trajectories in 800-μm-wide channels with and without adjoining iron channels. Using particle tracking velocimetry, a 54-fold higher lateral velocity was measured with iron channels, demonstrating that magnetic field gradients are significantly increased in the presence of the high-permeability channels (depicted in FIG. 14F).

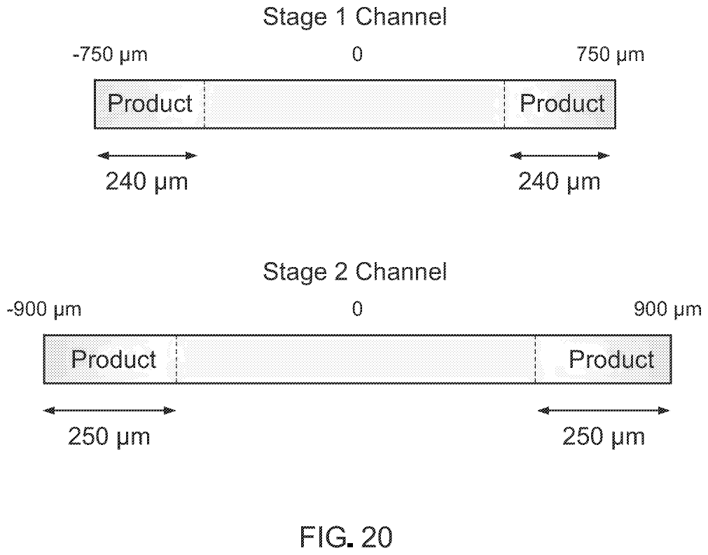

As depicted in FIGS. 14E and 14G, the magnetic gradient is maximal near the sidewalls of the sorting channels, and it decays progressively toward the center of the channels. Channels of the microchip can be designed to inertially arrange and sort cells in a small near-wall region in both stages. For example, in stage-1, channel width is 1500 μm, and the cutoff for deflection is set at 240 μm from the sidewalls. In stage-2, the channel width is 1800 μm, and the cutoff is set at 250 μm from the sidewall (depicted in FIG. 20). The height of the channels is kept constant at 60 μm.

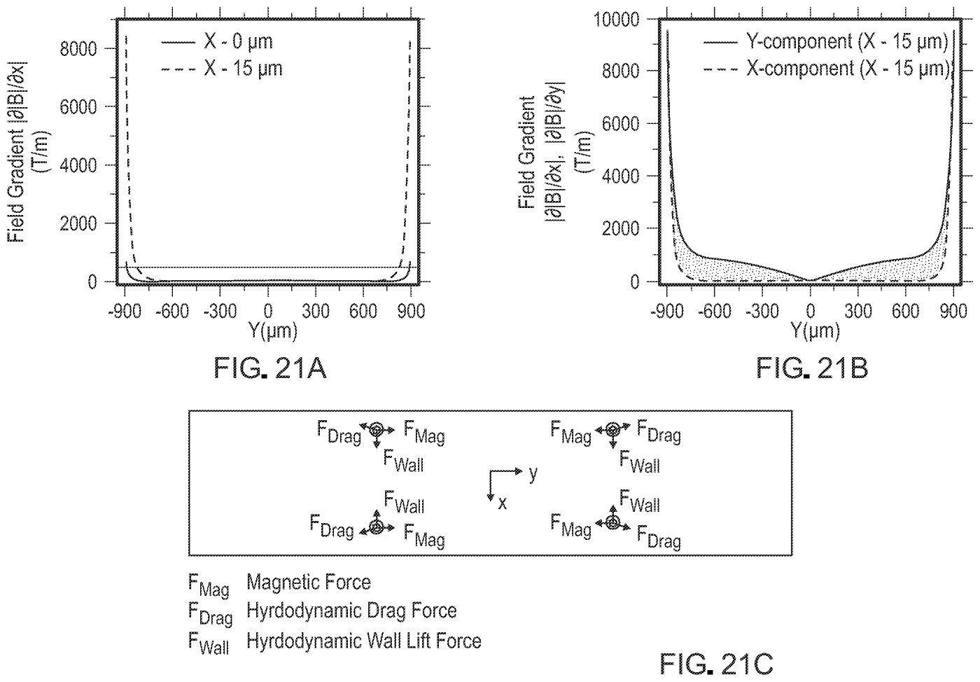

As depicted in FIG. 14G, an x-component of the magnetic field gradient is also present in the side-wall region and decays to ˜250 T/m within 100 μm from the sidewall and becomes negligible (<50 T/m) beyond 150 μm from the side wall (depicted in FIG. 21A). In comparison, the y-component of the magnetic field gradient is more than an order of magnitude stronger in the bulk of the sorting channel. This results in a magnetic force which is predominantly in lateral y-direction in the sorting channel. A cell undergoing magnetophoretic sorting primarily experiences a magnetic force toward the center of the channel in y-direction, wall lift force away from the top and bottom walls and a fluidic viscous drag force (depicted in FIG. 21C). The wall lift force prevents cells from touching the top and bottom walls as they migrate toward the center of the channel.

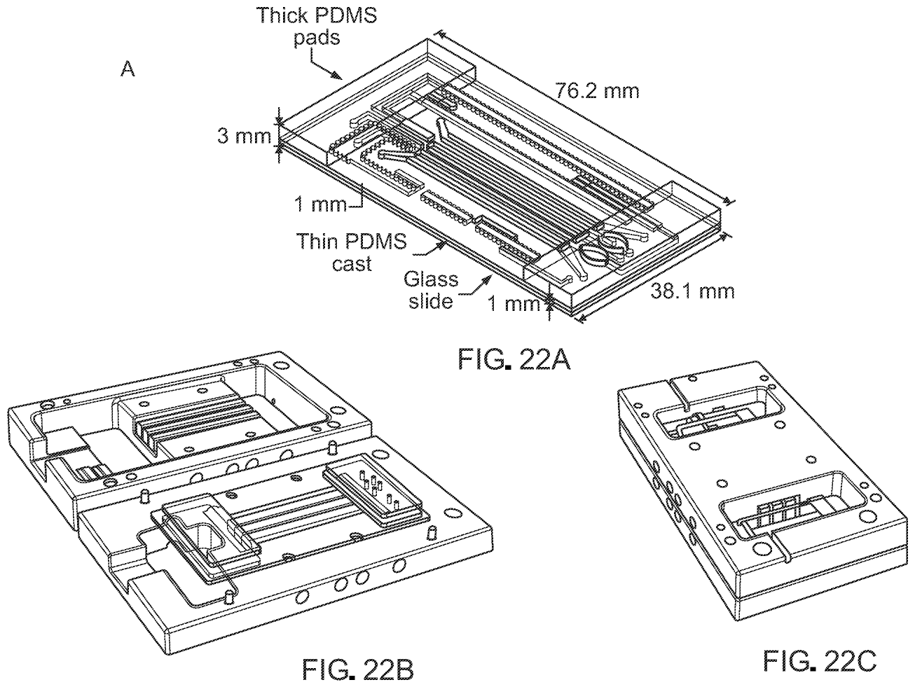

Deflection of cells in both sorting stages can be calculated using a laminar velocity profile for a low-aspect-ratio rectangular channel and the magnetic force expression. In stage-1, cells having greater than 10 beads along with most of the unbound magnetic beads are deflected to the waste port at a total flow rate of 168 mL/h, as depicted in FIG. 14H. In stage-2, cells are focused 100 μm away from the walls at a flow rate of 250 μL/min. In this stage, free beads and all cells with at least one bead attached to them are deflected, as depicted in FIG. 14I. FIGS. 22B and 22C show a manifold that can be used to hold a microchip and a set of magnets and allow the magnets to be brought close to the microchip.

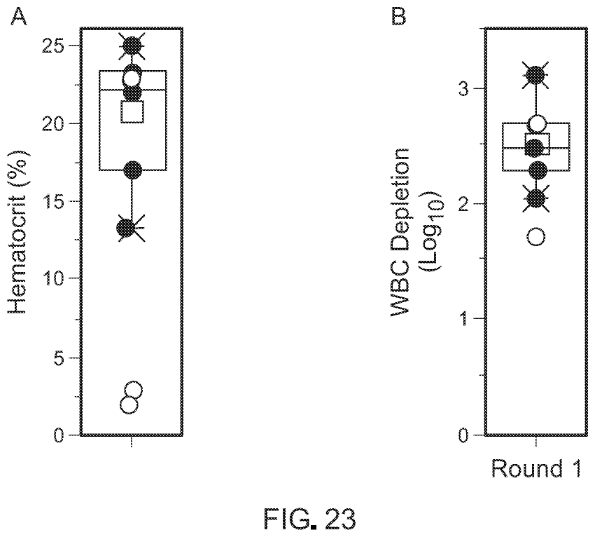

Sequential magnetic labeling can also be used to decrease the required number of magnetic beads/cell in some instance by approximately 91%. This was accomplished by initially labeling WBCs with 10 beads/cell, which results in depleting >99.5% of WBCs within ˜40 min by using the magnetic sorter (depicted in FIG. 23). The remaining 0.5% contaminating WBCs in the product were then relabeled with 125 beads/cell for 10 min and reprocessed through the magnetic sorter in less than 5 min for further removal.

c. Alignment-Free Magnetics

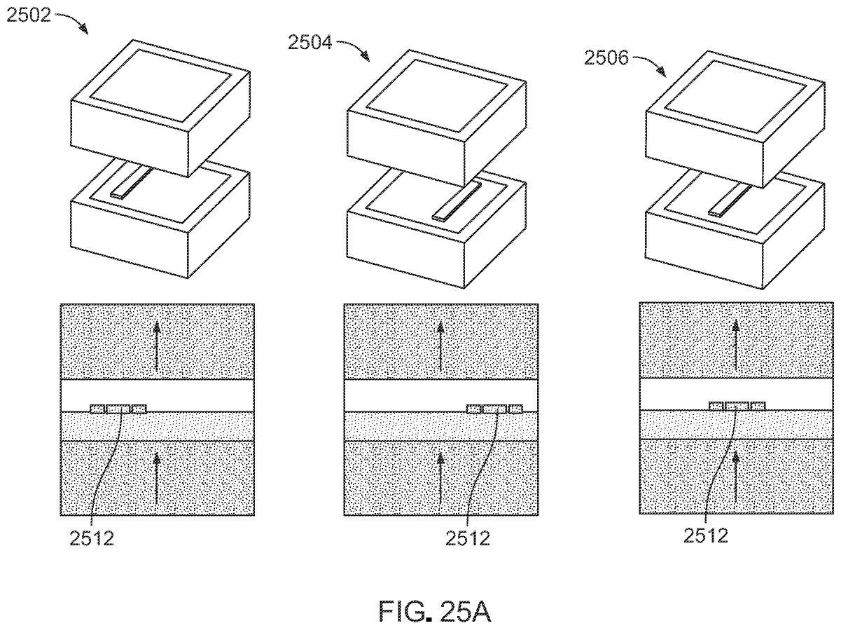

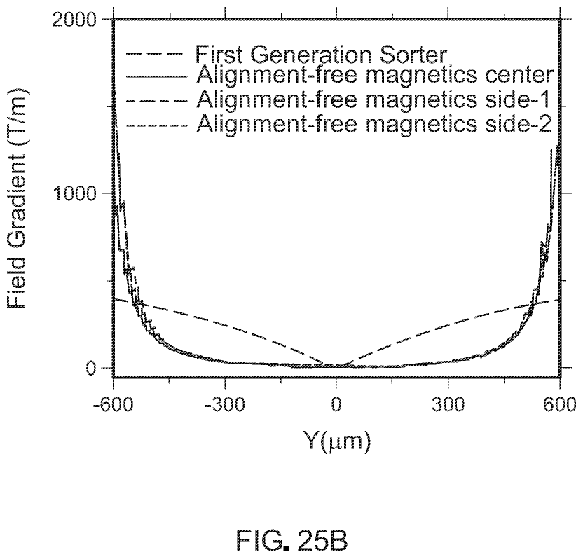

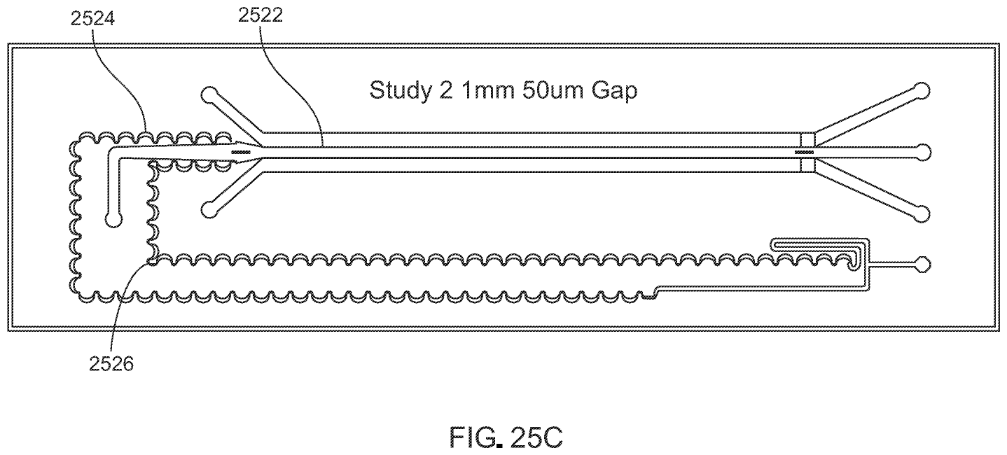

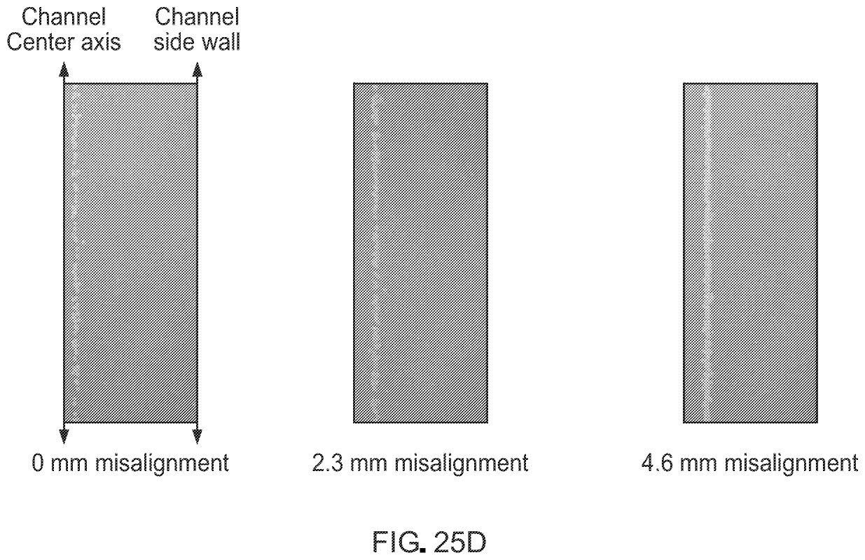

FIG. 25A shows different arrangements 2502, 2504, and 2506 of magnets that enable alignment-free magnetic field focusing using the microfluidic device depicted in FIG. 2A. In each arrangement, two external magnets (each with North pointing upwards) can be configured to provide alignment-free magnetic field focusing (i.e., magnetic field focusing that is unimpacted by positioning of a sample channel). In these examples, a sample channel 2512 is surrounded by two channels filled with high permeability material to provide near equivalent magnetic field gradients in the sample channel 2512 (e.g., for cell sorting), as shown in FIG. 25B. This contrasts with other configurations in which a lateral boundary between magnet pairs is carefully aligned with each other (top to bottom) and also to the sample channel, at least to a degree.

Alignment-free magnetic field focusing allows small misalignments to make little difference, allowing nearly arbitrary two-dimensional patterns of channels with high magnetic permeability material to be created in the plane between, for microfluidic applications. FIG. 25C depicts an example of a microfluidic device in which a magnetic sorting channel 2522 is fed by two inertial focusing modules 2524 and 2526. The microfluidic device is placed between two bulk magnets to allow sorting of target cells, as described throughout.

FIG. 25D are photographs of microfluidic channels with different arrangements of magnets. The photographs were collected using a high-speed video max-image stack (from ImageJ). Magnetic beads of 10 micron size were initially focused near to the channel side wall (right side; inlet not pictured). By the end of a magnetic sorting channel (as pictured), particles focused near to the midline (axis of bilateral symmetry). Exact position of particles within the channel shifted, even with nearly a 5-mm lateral offset of the microfluidic device with respect to the magnets (shown in the right). This result was expected given that magnetic field lines are orthogonal to the microfluidic device with very little lateral component.

d. Isolation of CTCs

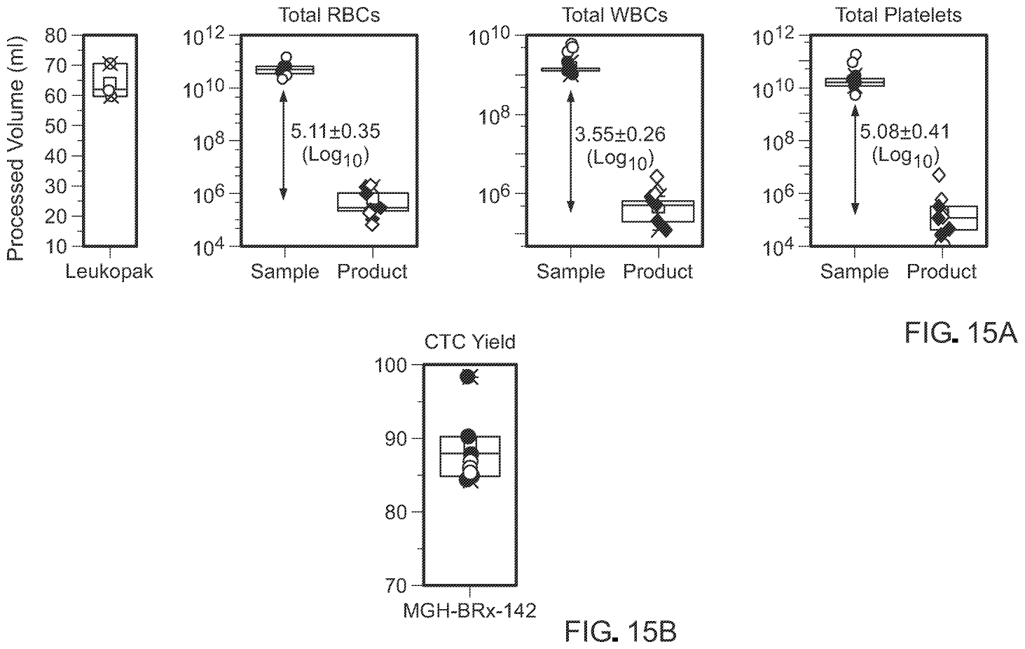

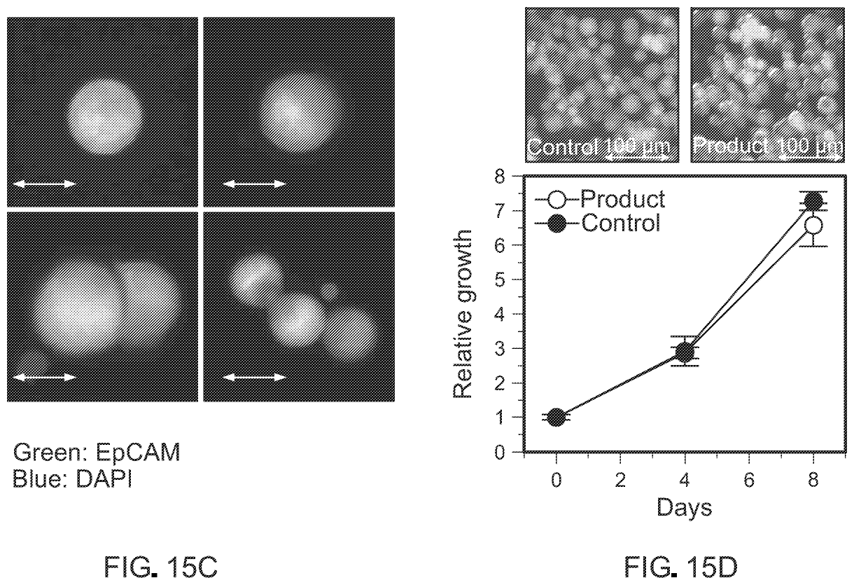

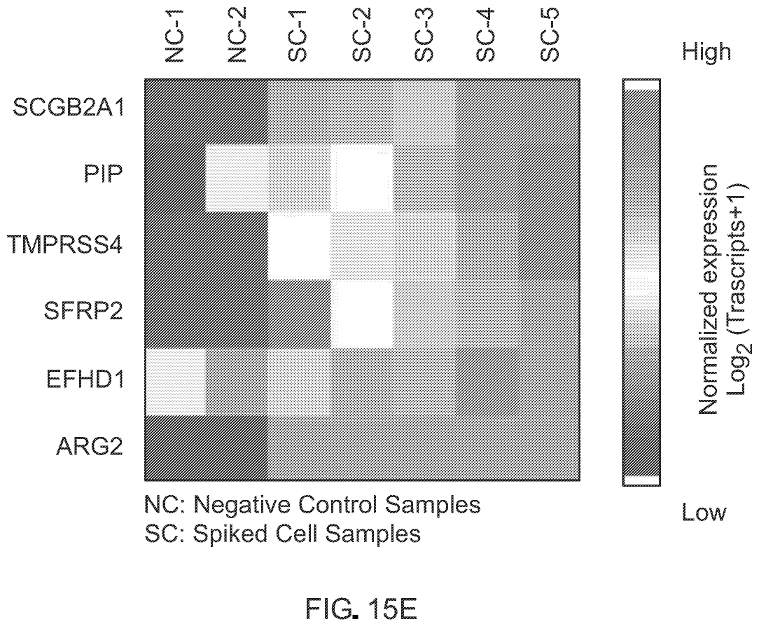

The performance of the permeability-enhanced ultra-high-throughput magnetic sorting device was evaluated by recovering CTCs spiked into three clinical leukapheresis samples (shown with dark data points in FIGS. 15A and 15B) and leukapheresis-mimic samples (samples spiked with ex vivo cultured CTCs; shown with light data points in FIGS. 15A and 15B).

The leukapheresis-mimic samples were produced by centrifuging approximately a unit of healthy donor blood (e.g., 400-500 mL whole blood) followed by extraction of the leukocyte-enriched layer. The samples on average contained approximately 1.42 billion WBCs, 56.5 billion RBCs, and 16.9 billion platelets (shown on the left graph of FIG. 15A). The mean volume of the samples was 24.5 mL and the WBC concentration varied from 39.5 to 82.6 million cells/mL at an average concentration of 58.4 million cells/mL. This is approximately 10-fold higher than whole blood.

The leukapheresis-mimic samples represented approximately a third of the clinical leukapheresis product in volume and the total number of nucleated cells while the concentration of WBCs is similar. One thousand green fluorescent protein (GFP)-expressing CTCs were spiked into these samples. The spiked CTCs were cultured from viable CTCs enriched from a blood sample of a patient with hormone receptor-positive breast cancer (MGH-BRx-142).

Using the approached described herein, 89.2±5.7% of spiked CTCs were recovered from the leukapheresis-mimic samples. Additionally, 99.998% of RBCs removed (4.88±0.37 log 10 depletion), 99.96% of WBCs were removed (3.35±0.17 log 10 depletion), and 99.998% platelets were removed (4.92±0.15 log 10 depletion). Performance of the inertial separation array module was separately quantified. The module removed 99.95% of RBCs (3.39±0.28 log 10 depletion) and 99.98% of platelets (3.83±0.19 log 10 depletion). The additional depletion of RBCs and platelets was achieved in stage-2 of the magnetic sorter, since RBCs and platelets are not inertially focused due to their smaller size and removed in the waste channels.

Three clinical leukapheresis samples containing 5.0±1.0 billion WBCs, 92.6±72.5 billion platelets, and 75.4±66.5 billion RBCs were also processed. WBCs in these samples were representative of a liter of whole blood. The average volume of these full samples was 64.2±4.6 mL into which 5,000 MGH-BRx-142 CTCs were spiked. This number of CTCs per Leukopak® is consistent with previous studies that processed 5% of clinical leukapheresis samples, calculating that, if technically feasible, screening of the entire Leukopak® would have produced >10,000 CTCs.

Depending on the apheresis operating conditions, the WBC concentration in these leukapheresis samples varied from 61 to 90 million cells/mL, while platelet concentrations ranged between 70 to 3043 million platelets/mL. Even though platelet and WBC concentrations were more than 10-fold higher than whole blood, two parallel sorting devices effectively removed 99.97% of WBCs (3.55±0.26 log 10 purification) while recovering 4305 CTCs out of the 5000 spiked CTCs (86.1±0.6% yield) at an average purity of 0.3% (depicted in FIG. 15B). The sorting device also depleted >99.999% of RBCs (5.11±0.35 log 10 purification) and >99.999% of platelets (5.08±0.41 log 10 purification), demonstrating a highly efficient microfluidic removal of contaminating RBCs and platelets (depicted in FIG. 15A). The isolation process also preserved the morphology as demonstrated by a gallery of EpCAM and DAPI stained CTCs shown FIG. 15C.

Post-isolation, a fraction of the product (40%) was tested in vitro culture to assess whether the sorting device had damaged the proliferative properties of isolated CTCs. FIG. 15D shows the relative in vitro growth of isolated CTCs in the product relative to control as measured by the amount of ATP present in cultured cells. Enriched CTCs from the product proliferated comparably with control samples.

An RNA-based droplet digital PCR (ddPCR) assay for absolute quantitation of tissue-lineage specific transcripts from CTCs in the background of normal blood cells was also performed. Results of the assay are depicted in FIG. 15E. The ddPCR assay confirmed the isolation of cells with intact RNA, suitable for molecular analyses.

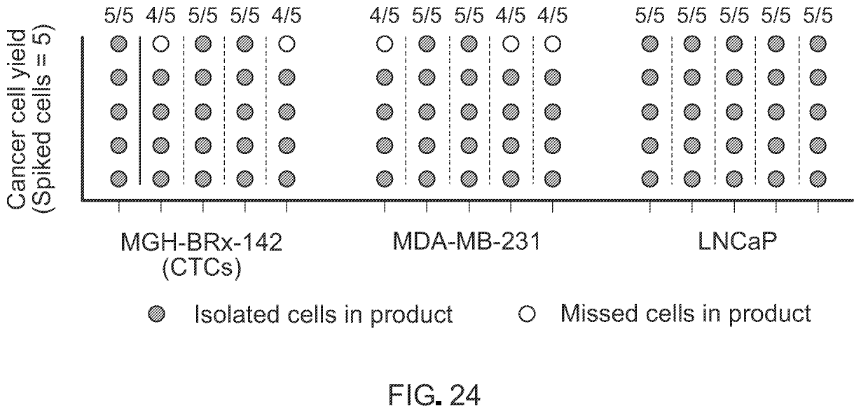

To mimic clinical situations in which CTC numbers are extremely low, such as early cancer detection applications, the sorting device was also tested by spiking five GFP-labeled cells from different cell lines including the breast CTC line MGH-BRx-142. The commonly studied breast cancer line MDA-MB-231 and the prostate cancer line LNCaP were also tested using the Leukopak® mimic assay. Results of these assays are depicted in FIG. 24. For precise quantitation of the number of cancer cells spiked into the Leukopak® mimics, cells were individually picked using single-cell micromanipulation and we conducted five independent CTC spiking experiments for each cell line. Using MGH-BRx-142, 4 of 5 spiked cells were recovered in two experiments, and 5 of 5 spiked cells were recovered in three additional experiments. Similarly, for MDA-MB-231, 4 of 5 cells were recovered in three spiking experiments and 5 of 5 spiked cells were recovered in two additional experiments. Using prostate LNCaP cells, 5 of 5 spiked cells were recovered in all five experiments.

### OTHER EMBODIMENTS

Several embodiments of the disclosure have been described. Nevertheless, it will be understood that various modifications may be made without departing from the spirit and scope of the disclosure. Accordingly, other embodiments are within the scope of the following claims.

1. A microfluidic device comprising:
a first particle enrichment module comprising a first microfluidic channel having a sample inlet at a first end of the first microfluidic channel, wherein the first microfluidic channel is configured to shift particles above a specific size to a first product outlet at a second end of the first microfluidic channel; and
a particle separation module comprising:
an array of islands in a second microfluidic channel, a buffer inlet in fluid communication with a first end of the second microfluidic channel, and a product inlet in fluid communication with the first product outlet and a first end of the second microfluidic channel, wherein:
the array of islands is arranged in one or more rows that extend along a longitudinal direction in a corresponding microfluidic channel,
each island in a row is spaced apart from an adjacent island in the row to form a siphoning channel, and
the array of islands is configured and arranged to shift portions of fluid through the siphoning channel between adjacent islands within a row to a waste outlet, and to shift particles above a specific size into a buffer flowing in the second microfluidic channel and to a second product outlet;
wherein the first particle enrichment module and the particle separation module are serially arranged such that a sample fluid having particles above the specific size flows from the first particle enrichment module to the particle separation module.

2. The microfluidic device of claim 1, further comprising:
a second particle enrichment module comprising a set of microfluidic channels that are each configured to shift particles above the specific size to first product outlets at respective second ends of the set of microfluidic channels.

3. The microfluidic device of claim 2, wherein:
the first particle enrichment module comprises:
a first array of islands in the first microfluidic channel, wherein:
the first array of islands is arranged in one or more rows that extend along a longitudinal direction in the first microfluidic channel,
each island in a row is spaced apart from an adjacent island in the row to form a siphoning channel, and
the first array of islands is configured and arranged to shift portions of fluid through the siphoning channel between adjacent islands within a row to a waste outlet at a second end of the first microfluidic channel, and to shift particles above a specific size to a first product outlet at the second end of the first microfluidic channel; and
the second particle enrichment module comprises:
multiple arrays of islands in a corresponding microfluidic channel included in the set of microfluidic channels, each array of islands having a sample inlet at a first end of the corresponding microfluidic channel, wherein:
each array included in the multiple arrays of islands is arranged in one or more rows that extend along a longitudinal direction in a corresponding microfluidic channel,
each island in a row is spaced apart from an adjacent island in the row to form a siphoning channel, and
each array included in the multiple arrays of islands is configured and arranged to shift portions of fluid through the siphoning channel between adjacent islands within a row to a waste outlet at a second end of the corresponding microfluidic channel, and to shift particles above a specific size to first product outlets at the second end of the corresponding microfluidic channel.

4. (canceled)

5. The microfluidic device of claim 3, wherein the multiple arrays of islands include four arrays of islands arranged in parallel such that a different portion of the sample fluid introduced into the microfluidic device flows through each of the four arrays of islands.

6. The microfluidic device of claim 1, wherein each island included in the array of islands has a width between 150 and 250 μm, a length between 200 and 800 μm, and a height between 100 and 200 μm.

7. The microfluidic device of claim 1, wherein each island included in the array of islands has a length-to-width ratio greater than 1.25.

8. (canceled)

9. A microfluidic device comprising:
a sorting channel arranged in a substrate and configured to flow a fluid sample comprising magnetized target entities;
a magnet placed underneath the substrate;
a permeability channel adjacent to a first side of the sorting channel and comprising a set of magnetic permeability particles; and
wherein the magnet and the set of magnetic permeability particles are configured to generate a deflecting magnetic field that causes a subset of magnetized target entities in the sorting channel to be deflected away from a first side of the sorting channel.

10. The microfluidic device of claim 9, wherein the set of magnetic permeability particles are configured to increase a gradient of the deflecting magnetic field.

11. The microfluidic device of claim 9, wherein the set of magnetic permeability particles are configured to change a direction of force exerted by the deflecting magnetic field on the magnetized target entities.

12. The microfluidic device of claim 9, wherein:
the deflecting magnetic field causes a second subset of magnetized target entities in the sorting channel to be deflected towards a second side of the sorting channel; and
the device further comprises:
a second permeability channel adjacent to the second side of the sorting channel opposite to the first side of the sorting channel, wherein the second permeability channel comprises a second subset of magnetic permeability particles.

13. The microfluidic device of claim 12, further comprising:
a first collection channel extending from the first side of the sorting channel such that the subset of magnetized target entities flows from the sorting channel to the first collection channel; and
a second collection channel extending from the second side of the sorting channel such that the second subset of magnetized target entities flow from the sorting channel to the second collection channel.

14. The microfluidic device of claim 13, further comprising:
a magnetic permeability strip placed underneath the sorting channel in the substrate adjacent to the magnet, wherein
the magnetic permeability strip extends longitudinally along a direction of fluid flow in the sorting channel, and
the magnetic permeability strip is configured to intensify the gradient of the deflecting magnetic field.

15. The microfluidic device of claim 9, further comprising a second sorting channel in the substrate and configured to receive a portion of the sample fluid flowing from the sorting channel.

16. The microfluidic device of claim 9, further comprising:
an inertial focusing channel in the substrate and comprising a set of asymmetric serpentine segments, wherein the inertial focusing channel is connected to the sorting channel such that a portion of the sample fluid flows from the inertial focusing channel to the first side of the first sorting channel.

17. (canceled)

18. The microfluidic device of claim 9, wherein:
the permeability channel includes an array of pillar structures sized to stabilize the set of magnetic permeability particles within the permeability channel.

19. (canceled)

20. The microfluidic device of claim 9, further comprising:
a second magnet placed above the substrate; and
wherein North poles of each of the magnet and the second magnet are facing an upward direction.

21. (canceled)

22. A method of concentrating and extracting particles from a sample fluid, the method comprising:
providing the sample fluid to a sorting channel of a microfluidic device;
providing a fluid containing a first set of magnetic permeability particles to a permeability channel, wherein the permeability channel is adjacent to a first side of the sorting channel; and
applying, using a magnet placed underneath the sorting channel, a deflecting magnetic field that causes a subset of magnetized target entities in the sorting channel to be deflected away from the first side of the sorting channel;
wherein the first set of magnetic permeability particles are configured to adjust the deflecting magnetic field generated by the magnet.

23. The method of claim 22, further comprising providing a buffer fluid to the sorting channel of the microfluidic device at a flow rate such that flow of the buffer fluid in the sorting channel maintains particle flow of the sample fluid to be directed towards the first side of the sorting channel.

24. The method of claim 22, further comprising:
providing a second fluid containing a second set of magnetic permeability particles to a second permeability channel, wherein the second permeability channel is adjacent to a second side of the sorting channel opposite to the first side of the sorting channel; and
applying, using the magnet, the deflecting magnetic field to cause a second subset of magnetized target entities in the sorting channel to be deflected towards a second side of the sorting channel.

25. The method of claim 24, further comprising passing, from the sorting channel and to a second sorting channel, a portion of the sample fluid flowing out of the sorting channel from the first side of the sorting channel and the second side of the first sorting channel, wherein the second sorting channel is adjacent to a side of the permeability channel that is opposite to a side of the permeability channel that is adjacent to the sorting channel.

26. The method of claim 22, wherein the sample fluid comprises a leukapheresis sample.
